(19) United States
(12) Patent Application Publication
(10) Pub. No.: US 2024/0009193 A1
(43) Pub. Date: Jan. 11, 2024

(54) METHODS OF TREATING ACHONDROPLASIA

(71) Applicant: QED Therapeutics, Inc., San Francisco, CA (US)

(72) Inventors: Riccardo Panicucci, Bluffton, SC (US); Susan Arangio, Los Altos, CA (US); Carl Dambkowski, San Mateo, CA (US); Michael Henderson, San Francisco, CA (US); Daniela Rogoff, Mountain View, CA (US); Michael Monteith, Raleigh, NC (US); Lihua Zhang, Cary, NC (US)

(21) Appl. No.: 18/257,450

(22) PCT Filed: Dec. 17, 2021
(86) PCT No.: PCT/US2021/064033

§ 371 Date: Jun. 14, 2023

Related U.S. Application Data
(60) Provisional application No. 63/127,576, filed on Dec. 18, 2020.

(51) Int. Cl. A61K 31/506; A61P 19/04; A61K 9/00; A61K 9/20

(52) U.S. Cl. CPC A61K 31/506; A61P 19/04; A61K 9/0053; A61K 9/2072; A61K 9/2018; A61K 9/2054; A61K 9/2027

(57) ABSTRACT

Provided herein are methods of treating a skeletal dysplasia such as hypochondroplasia or achondroplasia in a pediatric patient by administering to the patient infigratinib or a pharmaceutically acceptable salt thereof. Also provided are minitablets including infigratinib monophosphate and a pharmaceutically acceptable excipient for use in treating a skeletal dysplasia such as hypochondroplasia or achondroplasia in pediatric patients.

### CROSS-REFERENCE TO RELATED APPLICATION

The present application claims the benefit of and priority to U.S. Provisional Patent Application No. 63/127,576, filed Dec. 18, 2020, the disclosures of which are hereby incorporated by reference in their entireties for all purposes.

### BACKGROUND

Skeletal dysplasias can affect bone development, cartilage growth, and neurological functioning. Certain skeletal dysplasias, including achondroplasia (ACH) and hypochondroplasia (HCH), are forms of dwarfism. Achondroplasia (ACH) is the most common nonlethal form of skeletal dysplasia, affecting between 1 in 15,000 to 1 in 30,000 individuals (Horton W A, Hall J G, Hecht J T., Achondroplasia. Lancet, 2007, 370, 162-72; Waller D K, Correa A, Vo T M, Wang Y, Hobbs C, Langlois P H, et al., U S. Am. J. Med. Genet. A., 2008, 146A(18), 2385-2389). People with ACH have short stature, on average growing to approximately 4 feet (˜125 cm) in height, and are prone to significant co-morbidities including sleep apnea, chronic otitis media with conductive hearing loss, spinal stenosis, obesity, and in some cases, narrowing of the foramen magnum that can require urgent surgical intervention. HCH is similar to ACH and is characterized by less pronounced physical signs; however, HCH patients may also be impacted by greater neurological challenges including mild to moderate intellectual disabilities or learning problems.

ACH is characterized by defective endochondral ossification resulting from gain of function mutations in the fibroblast growth factor receptor (FGFR) 3 gene. These mutations, of which 99% are G380R mutations (Bellus G A, Hefferon T W, Ortiz de Luna R I, Hecht J T, Horton W A, Machado M, et al., Am. J. Hu. Genet., 56, 368-373, 1995), cause the receptor to be in a constitutively active state, disrupting the chondrocyte proliferation and differentiation in the growth plate and thereby inhibiting linear bone growth (Unger S, Bonafe L, Gouze D., Curr. Osteoporos. Rep., 2017, 15, 53-60). The key phenotype of ACH is disproportionate short stature with rhizomelia (shortened proximal limbs).

Approximately 80% of cases result from de novo mutations (Ornitz D M, Legeai-Mallet L., Dev. Dyn., 2017, 246, 291-309). Diagnosis typically occurs at birth, although it may be suspected on the basis of a late prenatal ultrasound.

There are no approved therapeutic interventions for ACH or HCH in North America, Europe, or Australia, and no widely accepted consensus about treatment (Unger). Current treatment options are nontargeted, ineffective, or painful interventions aimed at preventing or treating complications of ACH (FDA Background Document. Joint meeting of the Pediatric Advisory Committee and Endocrinologic and Metabolic Drugs Advisory Committee. 11 May 2018. Available at: https://www.fda.gov/downloads/AdvisoryCommittees/; Unger).

No clear growth effects have been shown after treatment with recombinant human growth hormone (r-hGH). An increase in growth velocity was noted (height increase from −5.0 to −4.0 standard deviations over 5 years), but no clear benefit was established for long-term treatment (Miccoli M, Bertelloni S, Massart F., Horm. Res. Paediatr., 2016, 86, 27-34); it is generally not a recommended treatment option for ACH (Horton; FDA).

Limb-lengthening procedures can provide 15-30 cm of additional height (˜20% increased length of bone segment), but the procedures are painful, often require repeat procedures, and have high complication rates (Horton). Furthermore, the growth rate of the growth plate can be disturbed by these procedures, and the cosmetic effect of long legs and short arms may not be suitable for some patients (FDA). Clinicians who consider limb lengthening now typically require psychological evaluations and require that children be old enough to provide assent for the procedure (Wright M J, Irving M D., Arch. Dis. Child., 2012, 97(2), 129-134).

Therefore, there remains an unmet need to develop novel therapeutic strategies for treating children with skeletal dysplasias such as achondroplasia and hypochondroplasia.

### SUMMARY

The present disclosure provides methods of treating skeletal dysplasias using an FGFR inhibitor, namely, infigratinib.

In one aspect, provided herein are methods of treating a skeletal dysplasia such as hypochondroplasia or achondroplasia in a pediatric patient in need thereof, the method generally comprises orally administering daily to the pediatric patient about 0.01 mg/kg to about 0.51 mg/kg of infigratinib, or a pharmaceutically acceptable salt thereof.

In certain embodiments, the method comprises administering to the pediatric patient about 0.01 mg/kg to about 0.3 mg/kg of infigratinib, or a pharmaceutically acceptable salt thereof. In certain embodiments, the method comprises administering to the pediatric patient about 0.01 mg/kg to about 0.15 mg/kg of infigratinib, or a pharmaceutically acceptable salt thereof. In certain embodiments, the method comprises administering to the pediatric patient about 0.015 mg/kg to about 0.13 mg/kg of infigratinib, or a pharmaceutically acceptable salt thereof. In certain embodiments, the method comprises administering to the pediatric patient about 0.016 mg/kg, about 0.032 mg/kg, about 0.064 mg/kg, about 0.128 mg/kg, about 0.256 mg/kg, or about 0.51 mg/kg of infigratinib, or a pharmaceutically acceptable salt thereof.

In another aspect, provided herein are methods of treating a pediatric patient suffering from a skeletal dysplasia such as hypochondroplasia or achondroplasia, the method generally comprising orally administering daily to the patient about 0.1 mg to about 8 mg of infigratinib, or a pharmaceutically acceptable salt thereof.

In certain embodiments, the method comprises administering to the pediatric patient about 1 mg to about 7 mg of infigratinib, or a pharmaceutically acceptable salt thereof.

In certain embodiments, the pediatric patient has a FGFR3 mutation. In certain embodiments, the FGFR3 mutation is a G380R mutation.

In certain embodiments, the infigratinib, or a pharmaceutically acceptable salt thereof, is administered as minitablets each having about 0.1 mg or about 1 mg of infigratinib, or a pharmaceutically acceptable salt thereof.

In certain embodiments, the infigratinib is present in the minitablets as infigratinib monophosphate. In certain embodiments, the infigratinib monophosphate is present as an anhydrous crystalline form. In certain embodiments, the anhydrous crystalline form of infigratinib monophosphate is characterized by an XRPD peak (2 theta) at 15.0°±0.2°.

In certain embodiments, after daily administration of the dose of infigratinib, or a pharmaceutically acceptable salt thereof, the pediatric patient exhibits increased height velocity relative to baseline. In certain embodiments, after daily administration of the dose of infigratinib, or a pharmaceutically acceptable salt thereof, the pediatric patient exhibits an increase in absolute height velocity.

In certain embodiments, after daily administration of the dose of infigratinib, or a pharmaceutically acceptable salt thereof, the pediatric patient exhibits an increase, relative to baseline, in an anthropometric measurement selected from the group consisting of standing height, sitting height, upper and lower arm length, thigh length, knee height, arm span, and combinations thereof. In certain embodiments, after daily administration of the dose of infigratinib, or a pharmaceutically acceptable salt thereof, the pediatric patient exhibits an absolute increase in an anthropometric measurement selected from the group consisting of standing height, sitting height, upper and lower arm length, thigh length, knee height, arm span, and combinations thereof.

In certain embodiments, after daily administration of the dose of infigratinib, or a pharmaceutically acceptable salt thereof, the pediatric patient exhibits a decrease, relative to baseline, in weight. In certain embodiments, after daily administration of the dose of infigratinib, or a pharmaceutically acceptable salt thereof, the pediatric patient exhibits an absolute decrease in weight.

In certain embodiments, after daily administration of the dose of infigratinib, or a pharmaceutically acceptable salt thereof, the pediatric patient exhibits a proportional increase, relative to baseline, in head circumference. In certain embodiments, after daily administration of the dose of infigratinib, or a pharmaceutically acceptable salt thereof, the pediatric patient exhibits an absolute proportional increase in head circumference.

In certain embodiments, after daily administration of the dose of infigratinib, or a pharmaceutically acceptable salt thereof, the pediatric patient exhibits a normalization, relative to baseline, of a body proportion measurement ratio. In certain embodiments, after daily administration of the dose of infigratinib, or a pharmaceutically acceptable salt thereof, the pediatric patient exhibits an absolute normalization of a body proportion measurement ratio. In certain embodiments, the body proportion measurement ratio is selected from the group consisting of upper to lower body segment ratio, upper arm to forearm ratio, upper leg to lower leg length ratio, arm span to standing height ratio, head circumference to standing height ratio, and combinations thereof.

In certain embodiments, after daily administration of the dose of infigratinib, or a pharmaceutically acceptable salt thereof, the pediatric patient exhibits an increase, relative to baseline, in a biomarker of bone turnover selected from the group consisting of type X collagen degradation fragment, collagen X marker, and combinations thereof.

In certain embodiments, after daily administration of the dose of infigratinib, or a pharmaceutically acceptable salt thereof, the pediatric patient exhibits an increase, relative to baseline, in mobility.

In certain embodiments, after daily administration of the dose of infigratinib, or a pharmaceutically acceptable salt thereof, the pediatric patient exhibits a decrease, relative to baseline, in the number of episodes of otitis media.

In certain embodiments, after daily administration of the dose of infigratinib, or a pharmaceutically acceptable salt thereof, the pediatric patient exhibits a decrease, relative to baseline, in the number of episodes and/or severity of sleep apnea.

In certain embodiments, after daily administration of the dose of infigratinib, or a pharmaceutically acceptable salt thereof, the pediatric patient exhibits an increase in quality of life, wherein quality of life is assessed using the Pediatric Quality of Life Inventory.

In certain embodiments, the pediatric patient is no more than 12 years of age. In certain embodiments, the pediatric patient is 3 to 11 years of age. In certain embodiments, the pediatric patient is less than 8 years of age. In certain embodiments, the pediatric patient is 8 years of age or older.

In another aspect, provided herein are minitablets for orally delivering about 1 mg of infigratinib, the minitablets generally comprise: infigratinib monophosphate, in an amount to deliver about 1 mg of infigratinib, and a pharmaceutically acceptable excipient.

In another aspect, provided herein are minitablets for orally delivering about 0.1 mg of infigratinib, the minitablets generally comprise: infigratinib monophosphate, in an amount to deliver about 0.1 mg of infigratinib, and a pharmaceutically acceptable excipient.

In certain embodiments, the pharmaceutically acceptable excipient is selected from the group consisting of mannitol, microcrystalline cellulose, a polyvinylpyrrolidone, a cross-linked polyvinylpyrrolidone, magnesium stearate, croscarmellose sodium, and combinations thereof.

In another aspect, provided herein are minitablets for oral delivery, the minitablets comprising: about 1% w/w to about 20% w/w of infigratinib monophosphate; and about 30% w/w to about 95% w/w of a filler.

In certain embodiments, the filler is selected from the group consisting of microcrystalline cellulose, lactose, mannitol, and combinations thereof. In certain embodiments, the filler comprises microcrystalline cellulose and mannitol.

In certain embodiments, the minitablet further comprises about 5% w/w to about 10% w/w of a binder. In certain embodiments, the binder is selected from the group consisting of a sugar, a gelatin, a natural gum, sorbitol, maltodextrin, an alginate, an alginate derivative, a polyvinylpyrrolidone, a cellulose, a cellulose derivative, and combinations thereof.

In certain embodiments, the minitablet further comprises about 5% w/w to about 10% w/w of a disintegrant. In certain embodiments, the disintegrant is selected from the group consisting of a starch, a starch derivative, a clay, a cross-linked cellulose, a cross-linked cellulose derivative, a cross-linked polyvinylpyrrolidone, and combinations thereof.

In another aspect, provided herein are minitablets for oral delivery, the minitablets comprising: about 1% w/w to about 20% w/w of infigratinib monophosphate; about 30% w/w to about 60% w/w of mannitol; and about 30% w/w to about 45% w/w of microcrystalline cellulose.

In certain embodiments, the minitablet comprises about 10% w/w to about 20% w/w of infigratinib monophosphate. In certain embodiments, the minitablet comprises about 1% w/w to about 5% w/w of infigratinib monophosphate.

In certain embodiments, the minitablet comprises about 30% w/w to about 45% w/w of mannitol. In certain embodiments, the minitablet comprises about 45% w/w to about 60% w/w of mannitol. In certain embodiments, the minitablet comprises about 35% w/w to about 40% w/w of microcrystalline cellulose.

In another aspect, provided herein are minitablets for oral delivery, the minitablets comprising: about 10% w/w to about 20% w/w of infigratinib monophosphate; about 30% w/w to about 45% w/w of mannitol; and about 30% w/w to about 40% w/w of microcrystalline cellulose.

In another aspect, provided herein are minitablets for oral delivery, the minitablets comprising: about 1% w/w to about 5% w/w of infigratinib monophosphate; about 45% w/w to about 60% w/w of mannitol; and about 30% w/w to about 40% w/w of microcrystalline cellulose.

In certain embodiments, the minitablet further comprises about 5% w/w to about 10% w/w of a polyvinylpyrrolidone. In certain embodiments, the minitablet further comprises about 5% w/w to about 10% w/w of a cross-linked polyvinylpyrrolidone. In certain embodiments, about 2% w/w to about 6% w/w of croscarmellose sodium.

In another aspect, provided herein are minitablets for oral delivery, the minitablets comprising: about 1.2 mg of infigratinib monophosphate; about 3 mg to about 4 mg of mannitol; and about 2 mg to about 4.4 mg of microcrystalline cellulose.

In certain embodiments, the minitablet further comprises about 0.6 mg to about 0.8 mg of a polyvinylpyrrolidone. In certain embodiments, the minitablet further comprises about 0.5 mg to about 0.7 mg of a cross-linked polyvinylpyrrolidone.

In another aspect, provided herein are minitablets for oral delivery, the minitablets comprising: about 0.12 mg of infigratinib monophosphate; about 3 mg to about 4 mg of mannitol; and about 2 mg to about 3 mg of microcrystalline cellulose.

In certain embodiments, the minitablet further comprises about 0.4 mg to about 0.6 mg of a polyvinylpyrrolidone. In certain embodiments, the minitablet further comprises about 0.3 mg to about 0.5 mg of a cross-linked polyvinylpyrrolidone. In certain embodiments, about 0.2 mg to about 0.4 mg of croscarmellose sodium.

### DETAILED DESCRIPTION

As generally described herein, the present disclosure provides methods of treating a skeletal dysplasia such as hypochondroplasia or achondroplasia in a pediatric patient in need thereof. The methods generally comprise administering an effective amount of infigratinib (e.g., milligrams per kilogram (mg/kg) to about 0.51 mg/kg) to the pediatric patient. In addition, a minitablet for delivering the effective amount of infigratinib to the pediatric patient is provided, for example, where the minitablet comprises infigratinib monophosphate and one or more pharmaceutically acceptable excipients (e.g., a filler, a binder, a disintegrant, a lubricant).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a excipient, inert or active, making the composition or formulation especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., infigratinib), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compound of the present invention are pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

The term “AUC” refers to the area under the time/plasma concentration curve after administration of the pharmaceutical composition. AUC_{0-infinity} denotes the area under the plasma concentration versus time curve from time 0 to infinity; AUC₀₋ₜ denotes the area under the plasma concentration versus time curve from time 0 to time t. It should be appreciated that AUC values can be determined by known methods in the art.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “subject” and “patient” are used interchangeably herein.

The term “Cₘₐₓ” refers to the maximum concentration of a therapeutic agent (e.g., infigratinib) in the blood (e.g., plasma) following administration of the pharmaceutical composition.

The term “tₘₐₓ” refers to the time in hours when Cₘₐₓ is achieved following administration of the pharmaceutical composition comprising a therapeutic agent (e.g., infigratinib).

As used herein, “solid dosage form” means a pharmaceutical dose(s) in solid form, e.g., tablets, capsules, granules, powders, sachets, reconstitutable powders, dry powder inhalers, and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). Infigratinib, or a pharmaceutically acceptable salt thereof, can be administered alone or can be co-administered to the patient (e.g., with another therapeutic agent). Co-administration is meant to include simultaneous or sequential administration of the compound individually or in combination with one or more additional agents, e.g., one or more additional therapeutic agents. Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”). Treatment may refer to any indicia of success in the amelioration or a disease, disorder, or condition, including any objective or subjective parameter such as abatement; remission; diminishing of symptoms or making the disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition.

Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

In general, an “effective amount” of a compound refers to an amount sufficient to elicit the desired biological response, e.g., to treat a skeletal dysplasia such as hypochondroplasia or achondroplasia. As will be appreciated by those of ordinary skill in this art, the effective amount of a compound of the disclosure may vary depending on such factors as the desired biological endpoint, the pharmacokinetics of the compound, the disease being treated, and the age, weight, health, and condition of the subject.

### Infigratinib

Infigratinib, as depicted in formula (I), is a selective and ATP-competitive pan-fibroblast growth factor receptor (FGFR) kinase inhibitor, also known as 3-(2,6-dichloro-3,5-dimethoxyphenyl)-1-{6-[4-(4-ethyl-1-piperazin-1-yl)phenylamino]-pyrimidinyl-4-yl}-1-methylurea. Infigratinib selectively inhibits the kinase activity of FGFR1, FGFR2, and FGFR3.

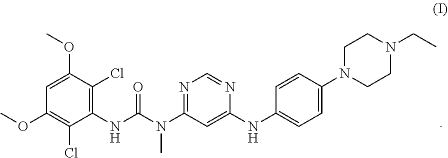 [SMILES: CCN1CCN(c2ccc(Nc3cc(N(C)C(=O)Nc4c(Cl)c(OC)cc(OC)c4Cl)ncn3)cc2)CC1]

A method of chemically synthesizing infigratinib (including Example 1 provided herein), several crystalline and amorphous forms of infigratinib (including the anhydrous crystalline monophosphate salt described herein) and methods of preparing said forms (including Example 2 provided herein) were described in U.S. Pat. No. 9,067,896, which is incorporated by reference in its entirety herein.

In one aspect, provided herein is infigratinib, or a pharmaceutically acceptable salt thereof, for the treatment of a skeletal dysplasia such as hypochondroplasia or achondroplasia in a pediatric patient in need thereof.

In certain embodiments, provided herein is a pharmaceutically acceptable salt of infigratinib for the treatment of a skeletal dysplasia such as hypochondroplasia or achondroplasia in a pediatric patient in need thereof. In some embodiments, the pharmaceutically acceptable salt of infigratinib is a monophosphate salt. The monophosphate salt of infigratinib may also be referred to as BGJ398, infigratinib phosphate, and infigratinib monophosphate.

In some embodiments, the monophosphate salt of infigratinib is an anhydrous crystalline monophosphate salt. In some embodiments, the anhydrous crystalline monophosphate salt has an X-ray powder diffraction (XRPD) pattern comprising a characteristic peak, in terms of 2θ, at about 15.0° or 15.0°±0.2°. In some embodiments, the X-ray powder diffraction pattern of the anhydrous crystalline monophosphate salt further comprises one or more characteristic peaks, in terms of 2θ, selected from peaks at about 13.7°±0.2°, about 16.8°±0.2°, about 21.3°±0.2° and about 22.4°±0.2°. In some embodiments, the X-ray powder diffraction pattern of the anhydrous crystalline monophosphate salt further comprises one or more characteristic peaks, in terms of selected from peaks at about 9.2°, about 9.6°, about 18.7°, about 20.0°, about 22.9°, and about 27.2°. In some embodiment, the anhydrous crystalline monophosphate salt has an XRPD pattern comprising at least three characteristic peaks, in terms of 2θ, selected from the peaks at about 13.7°, about 15°, about 16.8, about 21.3°, and about 22.4°. In some embodiments, the X-ray powder diffraction pattern for the anhydrous crystalline monophosphate salt may comprise one, two, three, four, five, six, seven, eight, nine, ten, or eleven characteristic peaks, in terms of 2θ, selected from the peaks at about 9.2°, about 9.6°, about 13.7°, about 15°, about 16.8°, about 18.7°, about 20.0°, about 21.3°, about 22.4°, about 22.9°, and about 27.2.

### Pharmaceutical Compositions

In one aspect, provided herein are pharmaceutical compositions comprising infigratinib, or a pharmaceutically acceptable salt thereof, and a pharmaceutically acceptable excipient, for the treatment of a skeletal dysplasia such as hypochondroplasia or achondroplasia in a pediatric patient in need thereof.

In various embodiments, provided herein are pharmaceutical compositions comprising infigratinib and a pharmaceutically acceptable excipient, for the treatment of a skeletal dysplasia such as hypochondroplasia or achondroplasia in a pediatric patient in need thereof.

In various embodiments, provided herein are pharmaceutical compositions comprising a pharmaceutically acceptable salt of infigratinib and a pharmaceutically acceptable excipient, for the treatment of a skeletal dysplasia such as hypochondroplasia or achondroplasia in a pediatric patient in need thereof. In certain embodiments, the pharmaceutically acceptable salt of infigratinib is infigratinib monophosphate.

In certain embodiments, the pharmaceutical composition is a minitablet. In certain embodiments, the minitablet is for oral delivery.

In various embodiments, provided herein are minitablets for orally delivering infigratinib, comprising infigratinib monophosphate and a pharmaceutically acceptable excipient. In certain embodiments, the minitablets orally deliver about 0.1 milligrams (mg) of infigratinib. In certain embodiments, the minitablets orally deliver about 1 mg of infigratinib.

In various embodiments, provided herein are minitablets for orally delivering about 0.1 mg of infigratinib, comprising infigratinib monophosphate and a pharmaceutically acceptable excipient.

In various embodiments, provided herein are minitablets for orally delivering about 1 mg of infigratinib, comprising infigratinib monophosphate and a pharmaceutically acceptable excipient.

In certain embodiments, the pharmaceutically acceptable excipient is selected from the group consisting of mannitol, microcrystalline cellulose, a polyvinylpyrrolidone, a cross-linked polyvinylpyrrolidone, magnesium stearate, croscarmellose sodium, and a combination thereof.

In various embodiments, provided herein are minitablets for oral delivery, comprising:
about 1% w/w to about 20% w/w of infigratinib monophosphate; and
about 30% w/w to about 95% w/w of a filler.

In certain embodiments, the minitablet comprises about 1% w/w to about 20% w/w, about 2% w/w to about 20% w/w, about 3% w/w to about 20% w/w, about 4% w/w to about 20% w/w, about 5% w/w to about 20% w/w, about 6% w/w to about 20% w/w, about 7% w/w to about 20% w/w, about 8% w/w to about 20% w/w, about 9% w/w to about 20% w/w, about 10% w/w to about 20% w/w, about 11% w/w to about 20% w/w, about 12% w/w to about 20% w/w, about 13% w/w to about 20% w/w, about 14% w/w to about 20% w/w, about 15% w/w to about 20% w/w, about 16% w/w to about 20% w/w, about 17% w/w to about 20% w/w, about 18% w/w to about 20% w/w, about 19% w/w to about 20% w/w, about 1% w/w to about 19% w/w, about 1% w/w to about 18% w/w, about 1% w/w to about 17% w/w, about 1% w/w to about 16% w/w, about 1% w/w to about 15% w/w, about 1% w/w to about 14% w/w, about 1% w/w to about 13% w/w, about 1% w/w to about 12% w/w, about 1% w/w to about 11% w/w, about 1% w/w to about 10% w/w, about 1% w/w to about 9% w/w, about 1% w/w to about 8% w/w, about 1% w/w to about 7% w/w, about 1% w/w to about 6% w/w, about 1% w/w to about 5% w/w, about 1% w/w to about 4% w/w, about 1% w/w to about 3% w/w, about 1% w/w to about 2% w/w, about 2% w/w to about 19% w/w, about 2% w/w to about 18% w/w, about 2% w/w to about 17% w/w, about 2% w/w to about 16% w/w, about 2% w/w to about 15% w/w, about 2% w/w to about 14% w/w, about 2% w/w to about 13% w/w, about 2% w/w to about 12% w/w, about 2% w/w to about 11% w/w, about 2% w/w to about 10% w/w, about 2% w/w to about 9% w/w, about 2% w/w to about 8% w/w, about 2% w/w to about 7% w/w, about 2% w/w to about 6% w/w, about 2% w/w to about 5% w/w, about 2% w/w to about 4% w/w, about 2% w/w to about 3% w/w, about 3% w/w to about 19% w/w, about 3% w/w to about 18% w/w, about 3% w/w to about 17% w/w, about 3% w/w to about 16% w/w, about 3% w/w to about 15% w/w, about 3% w/w to about 14% w/w, about 3% w/w to about 13% w/w, about 3% w/w to about 12% w/w, about 3% w/w to about 11% w/w, about 3% w/w to about 10% w/w, about 3% w/w to about 9% w/w, about 3% w/w to about 8% w/w, about 3% w/w to about 7% w/w, about 3% w/w to about 6% w/w, about 3% w/w to about 5% w/w, about 3% w/w to about 4% w/w, about 4% w/w to about 19% w/w, about 4% w/w to about 18% w/w, about 4% w/w to about 17% w/w, about 4% w/w to about 16% w/w, about 4% w/w to about 15% w/w, about 4% w/w to about 14% w/w, about 4% w/w to about 13% w/w, about 4% w/w to about 12% w/w, about 4% w/w to about 11% w/w, about 4% w/w to about 10% w/w, about 4% w/w to about 9% w/w, about 4% w/w to about 8% w/w, about 4% w/w to about 7% w/w, about 4% w/w to about 6% w/w, about 4% w/w to about 5% w/w, about 5% w/w to about 19% w/w, about 5% w/w to about 18% w/w, about 5% w/w to about 17% w/w, about 5% w/w to about 16% w/w, about 5% w/w to about 15% w/w, about 5% w/w to about 14% w/w, about 5% w/w to about 13% w/w, about 5% w/w to about 12% w/w, about 5% w/w to about 11% w/w, about 5% w/w to about 10% w/w, about 5% w/w to about 9% w/w, about 5% w/w to about 8% w/w, about 5% w/w to about 7% w/w, about 5% w/w to about 6% w/w, about 6% w/w to about 19% w/w, about 6% w/w to about 18% w/w, about 6% w/w to about 17% w/w, about 6% w/w to about 16% w/w, about 6% w/w to about 15% w/w, about 6% w/w to about 14% w/w, about 6% w/w to about 13% w/w, about 6% w/w to about 12% w/w, about 6% w/w to about 11% w/w, about 6% w/w to about 10% w/w, about 6% w/w to about 9% w/w, about 6% w/w to about 8% w/w, about 6% w/w to about 7% w/w, about 7% w/w to about 19% w/w, about 7% w/w to about 18% w/w, about 7% w/w to about 17% w/w, about 7% w/w to about 16% w/w, about 7% w/w to about 15% w/w, about 7% w/w to about 14% w/w, about 7% w/w to about 13% w/w, about 7% w/w to about 12% w/w, about 7% w/w to about 11% w/w, about 7% w/w to about 10% w/w, about 7% w/w to about 9% w/w, about 7% w/w to about 8% w/w, about 8% w/w to about 19% w/w, about 8% w/w to about 18% w/w, about 8% w/w to about 17% w/w, about 8% w/w to about 16% w/w, about 8% w/w to about 15% w/w, about 8% w/w to about 14% w/w, about 8% w/w to about 13% w/w, about 8% w/w to about 12% w/w, about 8% w/w to about 11% w/w, about 8% w/w to about 10% w/w, about 8% w/w to about 9% w/w, about 9% w/w to about 19% w/w, about 9% w/w to about 18% w/w, about 9% w/w to about 17% w/w, about 9% w/w to about 16% w/w, about 9% w/w to about 15% w/w, about 9% w/w to about 14% w/w, about 9% w/w to about 13% w/w, about 9% w/w to about 12% w/w, about 9% w/w to about 11% w/w, about 9% w/w to about 10% w/w, about 10% w/w to about 19% w/w, about 10% w/w to about 18% w/w, about 10% w/w to about 17% w/w, about 10% w/w to about 16% w/w, about 10% w/w to about 15% w/w, about 10% w/w to about 14% w/w, about 10% w/w to about 13% w/w, about 10% w/w to about 12% w/w, about 10% w/w to about 11% w/w, about 11% w/w to about 19% w/w, about 11% w/w to about 18% w/w, about 11% w/w to about 17% w/w, about 11% w/w to about 16% w/w, about 11% w/w to about 15% w/w, about 11% w/w to about 14% w/w, about 11% w/w to about 13% w/w, about 11% w/w to about 12% w/w, about 12% w/w to about 19% w/w, about 12% w/w to about 18% w/w, about 12% w/w to about 17% w/w, about 12% w/w to about 16% w/w, about 12% w/w to about 15% w/w, about 12% w/w to about 14% w/w, about 12% w/w to about 13% w/w, about 13% w/w to about 19% w/w, about 13% w/w to about 18% w/w, about 13% w/w to about 17% w/w, about 13% w/w to about 16% w/w, about 13% w/w to about 15% w/w, about 13% w/w to about 14% w/w, about 14% w/w to about 19% w/w, about 14% w/w to about 18% w/w, about 14% w/w to about 17% w/w, about 14% w/w to about 16% w/w, about 14% w/w to about 15% w/w, about 15% w/w to about 19% w/w, about 15% w/w to about 18% w/w, about 15% w/w to about 17% w/w, about 15% w/w to about 16% w/w, about 16% w/w to about 19% w/w, about 16% w/w to about 18% w/w, about 16% w/w to about 17% w/w, about 17% w/w to about 19% w/w, about 17% w/w to about 18% w/w, or about 18% w/w to about 19% w/w of infigratinib monophosphate. In certain embodiments, the minitablet comprises about 1% w/w to about 5% w/w of infigratinib monophosphate. In certain embodiments, the minitablet comprises about 10% w/w to about 20% w/w of infigratinib monophosphate.

In certain embodiments, the minitablet comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w of infigratinib monophosphate.

In certain embodiments, the minitablet comprises about 30% w/w to about 95% w/w, about 35% w/w to about 95% w/w, about 40% w/w to about 95% w/w, about 45% w/w to about 95% w/w, about 50% w/w to about 95% w/w, about 55% w/w to about 95% w/w, about 60% w/w to about 95% w/w, about 65% w/w to about 95% w/w, about 70% w/w to about 95% w/w, about 75% w/w to about 95% w/w, about 80% w/w to about 95% w/w, about 85% w/w to about 95% w/w, about 90% w/w to about 95% w/w, about 30% w/w to about 90% w/w, about 30% w/w to about 85% w/w, about 30% w/w to about 80% w/w, about 30% w/w to about 75% w/w, about 30% w/w to about 70% w/w, about 30% w/w to about 65% w/w, about 30% w/w to about 60% w/w, about 30% w/w to about 55% w/w, about 30% w/w to about 50% w/w, about 30% w/w to about 45% w/w, about 30% w/w to about 40% w/w, about 30% w/w to about 35% w/w, about 35% w/w to about 90% w/w, about 35% w/w to about 85% w/w, about 35% w/w to about 80% w/w, about 35% w/w to about 75% w/w, about 35% w/w to about 70% w/w, about 35% w/w to about 65% w/w, about 35% w/w to about 60% w/w, about 35% w/w to about 55% w/w, about 35% w/w to about 50% w/w, about 35% w/w to about 45% w/w, about 35% w/w to about 40% w/w, about 40% w/w to about 90% w/w, about 40% w/w to about 85% w/w, about 40% w/w to about 80% w/w, about 40% w/w to about 75% w/w, about 40% w/w to about 70% w/w, about 40% w/w to about 65% w/w, about 40% w/w to about 60% w/w, about 40% w/w to about 55% w/w, about 40% w/w to about 50% w/w, about 40% w/w to about 45% w/w, about 45% w/w to about 90% w/w, about 45% w/w to about 85% w/w, about 45% w/w to about 80% w/w, about 45% w/w to about 75% w/w, about 45% w/w to about 70% w/w, about 45% w/w to about 65% w/w, about 45% w/w to about 60% w/w, about 45% w/w to about 55% w/w, about 45% w/w to about 50% w/w, about 50% w/w to about 90% w/w, about 50% w/w to about 85% w/w, about 50% w/w to about 80% w/w, about 50% w/w to about 75% w/w, about 50% w/w to about 70% w/w, about 50% w/w to about 65% w/w, about 50% w/w to about 60% w/w, about 50% w/w to about 55% w/w, about 55% w/w to about 90% w/w, about 55% w/w to about 85% w/w, about 55% w/w to about 80% w/w, about 55% w/w to about 75% w/w, about 55% w/w to about 70% w/w, about 55% w/w to about 65% w/w, about 55% w/w to about 60% w/w, about 60% w/w to about 90% w/w, about 60% w/w to about 85% w/w, about 60% w/w to about 80% w/w, about 60% w/w to about 75% w/w, about 60% w/w to about 70% w/w, about 60% w/w to about 65% w/w, about 65% w/w to about 90% w/w, about 65% w/w to about 85% w/w, about 65% w/w to about 80% w/w, about 65% w/w to about 75% w/w, about 65% w/w to about 70% w/w, about 70% w/w to about 90% w/w, about 70% w/w to about 85% w/w, about 70% w/w to about 80% w/w, about 70% w/w to about 75% w/w, about 75% w/w to about 90% w/w, about 75% w/w to about 85% w/w, about 75% w/w to about 80% w/w, about 80% w/w to about 90% w/w, about 80% w/w to about 85% w/w, or about 80% w/w to about 90% w/w of a filler.

In certain embodiments, the minitablet comprises about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, or about 95% w/w of a filler.

In certain embodiments, the filler is selected from the group consisting of microcrystalline cellulose, lactose, mannitol, and a combination thereof.

In certain embodiments, the filler comprises microcrystalline cellulose and mannitol. In certain embodiments, the minitablet further comprises about 5% w/w to about 10% w/w, about 6% w/w to about 10% w/w, about 7% w/w to about 10% w/w, about 8% w/w to about 10% w/w, about 9% w/w to about 10% w/w, about 5% w/w to about 9% w/w, about 5% w/w to about 8% w/w, about 5% w/w to about 7% w/w, about 5% w/w to about 6% w/w, about 6% w/w to about 9% w/w, about 6% w/w to about 8% w/w, about 6% w/w to about 7% w/w, about 7% w/w to about 9% w/w, about 7% w/w to about 8% w/w, or about 8% w/w to about 9% w/w of a binder.

In certain embodiments, the minitablet further comprises about 5% w/w, about 6% w/w, about 7% w/w, about 8% w/w, about 9% w/w, or about 10% w/w of a binder.

In certain embodiments, the binder is selected from the group consisting of a sugar (e.g., glucose, sucrose), a gelatin, a natural gum (e.g., acacia, tragacanth), sorbitol, maltodextrin, an alginate (e.g., sodium alginate, an alginate derivative, a polyvinylpyrrolidone, a cellulose (e.g., microcrystalline cellulose), a cellulose derivative (e.g., methylcellulose, ethylcellulose, hydroxypropylcellulose, hydroxypropylmethylcellulose), and a combination thereof.

In certain embodiments, the minitablets further comprise about 5% w/w to about 10% w/w, about 6% w/w to about 10% w/w, about 7% w/w to about 10% w/w, about 8% w/w to about 10% w/w, about 9% w/w to about 10% w/w, about 5% w/w to about 9% w/w, about 5% w/w to about 8% w/w, about 5% w/w to about 7% w/w, about 5% w/w to about 6% w/w, about 6% w/w to about 9% w/w, about 6% w/w to about 8% w/w, about 6% w/w to about 7% w/w, about 7% w/w to about 9% w/w, about 7% w/w to about 8% w/w, or about 8% w/w to about 9% w/w of a disintegrant. In certain embodiments, the minitablets further comprise about 5% w/w to about 10% w/w of a disintegrant.

In certain embodiments, the minitablets further comprise about 5% w/w, about 6% w/w, about 7% w/w, about 8% w/w, about 9% w/w, or about 10% w/w of a disintegrant.

In certain embodiments, the disintegrant is selected from the group consisting of a starch (e.g., potato, maize, and corn starches), a starch derivative (e.g., low substituted carboxymethyl starches and pregelatinized starches), a clay (e.g., Veegum I-TV and bentonite), a cross-linked cellulose, a cross-linked cellulose derivative (e.g., cross-linked form of sodium carboxymethylcellulose), a cross-linked polyvinylpyrrolidone, and a combination thereof.

In various embodiments, provided herein are minitablets for oral delivery, comprising:
about 1% w/w to about 20% w/w of infigratinib monophosphate;
about 30% w/w to about 60% w/w of mannitol; and
about 30% w/w to about 45% w/w of microcrystalline cellulose.

In certain embodiments, the minitablet comprises about 30% w/w to about 60% w/w, about 35% w/w to about 60% w/w, about 40% w/w to about 60% w/w, about 45% w/w to about 60% w/w, about 50% w/w to about 60% w/w, about 55% w/w to about 60% w/w, about 30% w/w to about 55% w/w, about 30% w/w to about 50% w/w, about 30% w/w to about 45% w/w, about 30% w/w to about 40% w/w, about 30% w/w to about 35% w/w, about 35% w/w to about 55% w/w, about 35% w/w to about 50% w/w, about 35% w/w to about 45% w/w, about 35% w/w to about 40% w/w, about 40% w/w to about 55% w/w, about 40% w/w to about 50% w/w, about 40% w/w to about 45% w/w, about 45% w/w to about 55% w/w, about 45% w/w to about 50% w/w, or about 50% w/w to about 55% w/w of mannitol. In certain embodiments, the minitablet comprises about 30% w/w to about 45% w/w of mannitol. In certain embodiments, the minitablet comprises about 45% w/w to about 60% w/w of mannitol.

In certain embodiments, the minitablet comprises about 30% w/w, about 35% w/w, about 40% w/w, about 45% w/w, about 50% w/w, about 55% w/w, or about 60% w/w of mannitol.

In certain embodiments, the minitablet comprises about 30% w/w to about 45% w/w, about 35% w/w to about 45% w/w, about 40% w/w to about 45% w/w, about 30% w/w to about 40% w/w, about 30% w/w to about 35% w/w, or about 35% w/w to about 40% w/w of microcrystalline cellulose. In certain embodiments, the minitablet comprises about 35% w/w to about 40% w/w of microcrystalline cellulose.

In various embodiments, provided herein are minitablets for oral delivery, comprising:
about 10% w/w to about 20% w/w of infigratinib monophosphate;
about 30% w/w to about 45% w/w of mannitol; and
about 30% w/w to about 40% w/w of microcrystalline cellulose.

In various embodiments, provided herein are minitablets for oral delivery, comprising: about 1% w/w to about 5% w/w of infigratinib monophosphate;
about 45% w/w to about 60% w/w of mannitol; and
about 30% w/w to about 40% w/w of microcrystalline cellulose.

In certain embodiments, the minitablet further comprises about 5% w/w to about 10% w/w, about 6% w/w to about 10% w/w, about 7% w/w to about 10% w/w, about 8% w/w to about 10% w/w, about 9% w/w to about 10% w/w, about 5% w/w to about 9% w/w, about 5% w/w to about 8% w/w, about 5% w/w to about 7% w/w, about 5% w/w to about 6% w/w, about 6% w/w to about 9% w/w, about 6% w/w to about 8% w/w, about 6% w/w to about 7% w/w, about 7% w/w to about 9% w/w, about 7% w/w to about 8% w/w, or about 8% w/w to about 9% w/w of a polyvinylpyrrolidone. In certain embodiments, the minitablet further comprises about 5% w/w to about 10% w/w of a polyvinylpyrrolidone.

In certain embodiments, the minitablet further comprises about 5% w/w, about 6% w/w, about 7% w/w, about 8% w/w, about 9% w/w, or about 10% w/w of a polyvinylpyrrolidone.

In certain embodiments, the minitablet further comprises about 5% w/w to about 10% w/w, about 6% w/w to about 10% w/w, about 7% w/w to about 10% w/w, about 8% w/w to about 10% w/w, about 9% w/w to about 10% w/w, about 5% w/w to about 9% w/w, about 5% w/w to about 8% w/w, about 5% w/w to about 7% w/w, about 5% w/w to about 6% w/w, about 6% w/w to about 9% w/w, about 6% w/w to about 8% w/w, about 6% w/w to about 7% w/w, about 7% w/w to about 9% w/w, about 7% w/w to about 8% w/w, or about 8% w/w to about 9% w/w of a cross-linked polyvinylpyrrolidone. In certain embodiments, the minitablet further comprises about 5% w/w to about 10% w/w of a cross-linked polyvinylpyrrolidone.

In certain embodiments, the minitablet further comprises about 2% w/w to about 6% w/w, about 3% w/w to about 6% w/w, about 4% w/w to about 6% w/w, about 5% w/w to about 6% w/w, about 2% w/w to about 5% w/w, about 2% w/w to about 4% w/w, about 2% w/w to about 3% w/w, about 3% w/w to about 5% w/w, about 3% w/w to about 4% w/w, or about 4% w/w to about 5% w/w of croscarmellose sodium. In certain embodiments, the minitablet further comprises about 2% w/w to about 6% w/w of croscarmellose sodium.

In certain embodiments, the minitablet further comprises a lubricant. In certain embodiments, the minitablet further comprises about 0.25% w/w to about 1% w/w, about 0.5% w/w to about 1% w/w, about 0.75% w/w to about 1% w/w, about 0.25% w/w to about 0.75% w/w, about 0.25% w/w to about 0.5% w/w, or about 0.5% w/w to about 0.75% w/w of a lubricant. In certain embodiments, the minitablet further comprises about 0.5% w/w to about 0.75% w/w of a lubricant.

In certain embodiments, the minitablet further comprises about 0.25% w/w, about 0.3% w/w, about 0.35% w/w, about 0.4% w/w, about 0.45% w/w, about 0.5% w/w, about 0.55% w/w, about 0.6% w/w, about 0.65% w/w, about 0.7% w/w, about 0.75% w/w, about 0.8% w/w, about w/w, about 0.9% w/w, about 0.95% w/w, or about 1% w/w of a lubricant. In certain embodiments, the minitablet further comprises about 0.5% w/w of a lubricant. In certain embodiments, the minitablet further comprises about 0.75% w/w of a lubricant. In certain embodiments, the minitablet further comprises about 1% w/w of a lubricant.

In certain embodiments, the lubricant is magnesium stearate.

In certain embodiments, the minitablet further comprises a colorant.

In various embodiments, provided herein are minitablets for oral delivery, comprising:
about 1.2 mg of infigratinib monophosphate;
about 3 mg to about 4 mg of mannitol; and
about 2 mg to about 4.4 mg of microcrystalline cellulose.

In various embodiments, provided herein are minitablets for oral delivery, comprising:
about 0.12 mg of infigratinib monophosphate;
about 3 mg to about 4 mg of mannitol; and
about 2 mg to about 3 mg of microcrystalline cellulose.

In certain embodiments, the minitablet comprises about 3 mg to about 4 mg, about 3.2 mg to about 4 mg, about 3.4 mg to about 4 mg, about 3.6 mg to about 4 mg, about 3.8 mg to about 4 mg, about 3 mg to about 3.8 mg, about 3 mg to about 3.6 mg, about 3 mg to about 3.4 mg, about 3 mg to about 3.2 mg, about 3.2 mg to about 3.8 mg, about 3.2 mg to about 3.6 mg, about 3.2 mg to about 3.4 mg, about 3.4 mg to about 3.8 mg, about 3.4 mg to about 3.6 mg, or about 3.6 mg to about 3.8 mg of mannitol.

In certain embodiments, the minitablet comprises about 3 mg, about 3.1 mg, about 3.2 mg, about 3.3 mg, about 3.4 mg, about 3.5 mg, about 3.6 mg, about 3.7 mg, about 3.8 mg, about 3.9 mg, or about 4 mg of mannitol.

In certain embodiments, the minitablet comprises about 2 mg to about 4.4 mg, about 2.2 mg to about 4.4 mg, about 2.4 mg to about 4.4 mg, about 2.6 mg to about 4.4 mg, about 2.8 mg to about 4.4 mg, about 3 mg to about 4.4 mg, about 3.2 mg to about 4.4 mg, about 3.4 mg to about 4.4 mg, about 3.6 mg to about 4.4 mg, about 3.8 mg to about 4.4 mg, about 4 mg to about 4.4 mg, about 4.2 mg to about 4.4 mg, about 2 mg to about 4.2 mg, about 2 mg to about 4 mg, about 2 mg to about 3.8 mg, about 2 mg to about 3.6 mg, about 2 mg to about 3.4 mg, about 2 mg to about 3.2 mg, about 2 mg to about 3 mg, about 2 mg to about 2.8 mg, about 2 mg to about 2.6 mg, about 2 mg to about 2.4 mg, about 2 mg to about 2.2 mg, about 2.2 mg to about 4.2 mg, about 2.2 mg to about 4 mg, about 2.2 mg to about 3.8 mg, about 2.2 mg to about 3.6 mg, about 2.2 mg to about 3.4 mg, about 2.2 mg to about 3.2 mg, about 2.2 mg to about 3 mg, about 2.2 mg to about 2.8 mg, about 2.2 mg to about 2.6 mg, about 2.2 mg to about 2.4 mg, about 2.4 mg to about 4.2 mg, about 2.4 mg to about 4 mg, about 2.4 mg to about 3.8 mg, about 2.4 mg to about 3.6 mg, about 2.4 mg to about 3.4 mg, about 2.4 mg to about 3.2 mg, about 2.4 mg to about 3 mg, about 2.4 mg to about 2.8 mg, about 2.4 mg to about 2.6 mg, about 2.6 mg to about 4.2 mg, about 2.6 mg to about 4 mg, about 2.6 mg to about 3.8 mg, about 2.6 mg to about 3.6 mg, about 2.6 mg to about 3.4 mg, about 2.6 mg to about 3.2 mg, about 2.6 mg to about 3 mg, about 2.6 mg to about 2.8 mg, about 2.8 mg to about 4.2 mg, about 2.8 mg to about 4 mg, about 2.8 mg to about 3.8 mg, about 2.8 mg to about 3.6 mg, about 2.8 mg to about 3.4 mg, about 2.8 mg to about 3.2 mg, about 2.8 mg to about 3.0 mg, about 3 mg to about 4.2 mg, about 3 mg to about 4 mg, about 3 mg to about 3.8 mg, about 3 mg to about 3.6 mg, about 3 mg to about 3.4 mg, about 3 mg to about 3.2 mg, about 3.2 mg to about 4.2 mg, about 3.2 mg to about 4 mg, about 3.2 mg to about 3.8 mg, about 3.2 mg to about 3.6 mg, about 3.2 mg to about 3.4 mg, about 3.4 mg to about 4.2 mg, about 3.4 mg to about 4 mg, about 3.4 mg to about 3.8 mg, about 3.4 mg to about 3.6 mg, about 3.6 mg to about 4.2 mg, about 3.6 mg to about 4 mg, about 3.6 mg to about 3.8 mg, about 3.8 mg to about 4.2 mg, about 3.8 mg to about 4 mg, or about 4 mg to about 4.2 mg of microcrystalline cellulose.

In certain embodiments, the minitablet comprises about 2 mg, about 2.2 mg, about 2.4 mg, about 2.6 mg, about 2.8 mg, about 3 mg, about 3.2 mg, about 3.4 mg, about 3.6 mg, about 3.8 mg, about 4 mg, about 4.2 mg, or about 4.4 mg of microcrystalline cellulose.

In certain embodiments, the minitablet further comprises about 0.4 mg to about 0.6 mg, about 0.45 mg to about 0.6 mg, about 0.5 mg to about 0.6 mg, about 0.55 mg to about 0.6 mg, about 0.4 mg to about 0.55 mg, about 0.4 mg to about 0.5 mg, about 0.4 mg to about 0.45 mg, about 0.45 mg to about 0.55 mg, about 0.45 mg to about 0.5 mg, or about 0.5 mg to about 0.55 mg of a polyvinylpyrrolidone. In certain embodiments, the minitablet further comprises about 0.4 mg to about 0.6 mg of a polyvinylpyrrolidone.

In certain embodiments, the minitablet further comprises about 0.4 mg, about 0.42 mg, about 0.44 mg, about 0.46 mg, about 0.48 mg, about 0.5 mg, about 0.52 mg, about 0.54 mg, about 0.56 mg, about 0.58 mg, or about 0.6 mg of a polyvinylpyrrolidone.

In certain embodiments, the minitablet further comprises about 0.3 mg to about 0.5 mg, about 0.35 mg to about 0.5 mg, about 0.4 mg to about 0.5 mg, about 0.45 mg to about 0.5 mg, about 0.3 mg to about 0.45 mg, about 0.3 mg to about 0.4 mg, about 0.3 mg to about 0.35 mg, about 0.35 mg to about 0.45 mg, about 0.35 mg to about 0.4 mg, or about 0.4 mg to about 0.45 mg of a cross-linked polyvinylpyrrolidone. In certain embodiments, the minitablet further comprises about 0.3 mg to about 0.5 mg of a cross-linked polyvinylpyrrolidone.

In certain embodiments, the minitablet further comprises about 0.3 mg, about 0.32 mg, about 0.34 mg, about 0.36 mg, about 0.38 mg, about 0.4 mg, about 0.42 mg, about 0.44 mg, about 0.46 mg, about 0.48 mg, or about 0.5 mg of a cross-linked polyvinylpyrrolidone.

In certain embodiments, the minitablet further comprises about 0.2 mg to about 0.4 mg, about 0.25 mg to about 0.4 mg, about 0.3 mg to about 0.4 mg, about 0.35 mg to about 0.4 mg, about 0.2 mg to about 0.35 mg, about 0.2 mg to about 0.3 mg, about 0.2 mg to about 0.25 mg, about 0.25 mg to about 0.35 mg, about 0.25 mg to about 0.3 mg, or about 0.3 mg to about 0.35 mg of croscarmellose sodium. In certain embodiments, the minitablet further comprises about 0.2 mg to about 0.4 mg of croscarmellose sodium.

In certain embodiments, the minitablet further comprises about 0.2 mg, about 0.22 mg, about 0.24 mg, about 0.26 mg, about 0.28 mg, about 0.3 mg, about 0.32 mg, about 0.34 mg, about 0.36 mg, about 0.38 mg, or about 0.4 mg of croscarmellose sodium.

In certain embodiments, the minitablet further comprises about 0.03 mg to about 0.09 mg, about 0.04 mg to about 0.09 mg, about 0.05 mg to about 0.09 mg, about 0.06 mg to about 0.09 mg, about 0.07 mg to about 0.09 mg, about 0.08 mg to about 0.09 mg, about 0.03 mg to about 0.08 mg, about 0.03 mg to about 0.07 mg, about 0.03 mg to about 0.06 mg, about 0.03 mg to about 0.05 mg, about 0.03 mg to about 0.04 mg, about 0.04 mg to about 0.08 mg, about 0.04 mg to about 0.07 mg, about 0.04 mg to about 0.06 mg, about 0.04 mg to about 0.05 mg, about 0.05 mg to about 0.08 mg, about 0.05 mg to about 0.07 mg, about 0.05 mg to about 0.06 mg, about 0.06 mg to about 0.08 mg, about 0.06 mg to about 0.07 mg, or about 0.07 mg to about 0.08 mg of magnesium stearate.

In certain embodiments, the minitablet further comprises about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, or about 0.1 mg of magnesium stearate. In certain embodiments, the minitablet further comprises about 0.035 mg of magnesium stearate. In certain embodiments, the minitablet further comprises about 0.055 mg of magnesium stearate. In certain embodiments, the minitablet further comprises about 0.07 mg of magnesium stearate. In certain embodiments, the minitablet further comprises about 0.085 mg of magnesium stearate.

In certain embodiments, the infigratinib monophosphate is present as an anhydrous crystalline form. In certain embodiments, the anhydrous crystalline form of infigratinib monophosphate is characterized by an XRPD peak (2 theta) at 15.0°±0.2°.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

Skeletal dysplasias affecting bone development and other functions include, for example, achondroplasia and hypochondroplasia. Achondroplasia (ACH) is the most common nonlethal form of skeletal dysplasia and is characterized by defective endochondral ossification resulting from gain of function mutations in the fibroblast growth factor receptor (FGFR) 3 gene.

In one aspect, provided herein are methods of treating a skeletal dysplasia such as hypochondroplasia or achondroplasia in a pediatric patient in need thereof.

In various embodiments, provided herein are methods of treating a skeletal dysplasia such as hypochondroplasia or achondroplasia in a pediatric patient in need thereof, comprising orally administering daily to the pediatric patient about 0.01 mg/kg to about 0.51 mg/kg of infigratinib, or a pharmaceutically acceptable salt thereof.

In certain embodiments, the method comprises administering to the pediatric patient about 0.01 mg/kg to about 0.51 mg/kg, about 0.05 mg/kg to about 0.51 mg/kg, about 0.1 mg/kg to about 0.51 mg/kg, about 0.15 mg/kg to about 0.51 mg/kg, about 0.2 mg/kg to about 0.51 mg/kg, about 0.25 mg/kg to about 0.51 mg/kg, about 0.3 mg/kg to about 0.51 mg/kg, about 0.35 mg/kg to about 0.51 mg/kg, about 0.4 mg/kg to about 0.51 mg/kg, about 0.45 mg/kg to about mg/kg, about 0.01 mg/kg to about 0.45 mg/kg, about 0.01 mg/kg to about 0.4 mg/kg, about mg/kg to about 0.35 mg/kg, about 0.01 mg/kg to about 0.3 mg/kg, about 0.01 mg/kg to about 0.25 mg/kg, about 0.01 mg/kg to about 0.2 mg/kg, about 0.01 mg/kg to about 0.15 mg/kg, about 0.01 mg/kg to about 0.1 mg/kg, about 0.01 mg/kg to about 0.05 mg/kg, about 0.05 mg/kg to about 0.45 mg/kg, about 0.05 mg/kg to about 0.4 mg/kg, about 0.05 mg/kg to about 0.35 mg/kg, about 0.05 mg/kg to about 0.3 mg/kg, about 0.05 mg/kg to about 0.25 mg/kg, about 0.05 mg/kg to about 0.2 mg/kg, about 0.05 mg/kg to about 0.15 mg/kg, about 0.05 mg/kg to about 0.1 mg/kg, about 0.1 mg/kg to about 0.45 mg/kg, about 0.1 mg/kg to about 0.4 mg/kg, about 0.1 mg/kg to about 0.35 mg/kg, about 0.1 mg/kg to about 0.3 mg/kg, about 0.1 mg/kg to about 0.25 mg/kg, about 0.1 mg/kg to about 0.2 mg/kg, about 0.1 mg/kg to about 0.15 mg/kg, about 0.15 mg/kg to about 0.45 mg/kg, about 0.15 mg/kg to about 0.4 mg/kg, about 0.15 mg/kg to about mg/kg, about 0.15 mg/kg to about 0.3 mg/kg, about 0.15 mg/kg to about 0.25 mg/kg, about mg/kg to about 0.2 mg/kg, about 0.2 mg/kg to about 0.45 mg/kg, about 0.2 mg/kg to about mg/kg, about 0.2 mg/kg to about 0.35 mg/kg, about 0.2 mg/kg to about 0.3 mg/kg, about 0.2 mg/kg to about 0.25 mg/kg, about 0.25 mg/kg to about 0.45 mg/kg, about 0.25 mg/kg to about mg/kg, about 0.25 mg/kg to about 0.35 mg/kg, about 0.25 mg/kg to about 0.3 mg/kg, about mg/kg to about 0.45 mg/kg, about 0.3 mg/kg to about 0.4 mg/kg, about 0.3 mg/kg to about mg/kg, about 0.35 mg/kg to about 0.45 mg/kg, about 0.35 mg/kg to about 0.4 mg/kg, or about 0.4 mg/kg to about 0.45 mg/kg of infigratinib, or a pharmaceutically acceptable salt thereof. In certain embodiments, the method comprises administering to the pediatric patient about 0.01 mg/kg to about 0.15 mg/kg of infigratinib, or a pharmaceutically acceptable salt thereof. In certain embodiments, the method comprises administering to the pediatric patient about 0.01 mg/kg to about 0.3 mg/kg of infigratinib, or a pharmaceutically acceptable salt thereof.

In certain embodiments, the method comprises administering to the pediatric patient about 0.01 mg/kg to about 0.15 mg/kg, about 0.015 mg/kg to about 0.15 mg/kg, about 0.02 mg/kg to about 0.15 mg/kg, about 0.025 mg/kg to about 0.15 mg/kg, about 0.03 mg/kg to about mg/kg, about 0.05 mg/kg to about 0.15 mg/kg, about 0.07 mg/kg to about 0.15 mg/kg, about 0.09 mg/kg to about 0.15 mg/kg, about 0.11 mg/kg to about 0.15 mg/kg, about 0.13 mg/kg to about 0.15 mg/kg, about 0.01 mg/kg to about 0.13 mg/kg, about 0.01 mg/kg to about 0.11 mg/kg, about 0.01 mg/kg to about 0.09 mg/kg, about 0.01 mg/kg to about 0.07 mg/kg, about mg/kg to about 0.05 mg/kg, about 0.01 mg/kg to about 0.03 mg/kg, about 0.01 mg/kg to about 0.025 mg/kg, about 0.01 mg/kg to about 0.02 mg/kg, about 0.01 mg/kg to about 0.015 mg/kg, about 0.015 mg/kg to about 0.13 mg/kg, about 0.015 mg/kg to about 0.11 mg/kg, about mg/kg to about 0.09 mg/kg, about 0.015 mg/kg to about 0.07 mg/kg, about 0.015 mg/kg to about 0.05 mg/kg, about 0.015 mg/kg to about 0.03 mg/kg, about 0.015 mg/kg to about 0.025 mg/kg, about 0.015 mg/kg to about 0.02 mg/kg, about 0.02 mg/kg to about 0.13 mg/kg, about mg/kg to about 0.11 mg/kg, about 0.02 mg/kg to about 0.09 mg/kg, about 0.02 mg/kg to about 0.07 mg/kg, about 0.02 mg/kg to about 0.05 mg/kg, about 0.02 mg/kg to about 0.03 mg/kg, about 0.02 mg/kg to about 0.025 mg/kg, about 0.025 mg/kg to about 0.13 mg/kg, about mg/kg to about 0.11 mg/kg, about 0.025 mg/kg to about 0.09 mg/kg, about 0.025 mg/kg to about 0.07 mg/kg, about 0.025 mg/kg to about 0.05 mg/kg, about 0.025 mg/kg to about 0.03 mg/kg, about 0.03 mg/kg to about 0.13 mg/kg, about 0.03 mg/kg to about 0.11 mg/kg, about mg/kg to about 0.09 mg/kg, about 0.03 mg/kg to about 0.07 mg/kg, about 0.03 mg/kg to about 0.05 mg/kg, about 0.05 mg/kg to about 0.13 mg/kg, about 0.05 mg/kg to about 0.11 mg/kg, about 0.05 mg/kg to about 0.09 mg/kg, about 0.05 mg/kg to about 0.07 mg/kg, about 0.07 mg/kg to about 0.13 mg/kg, about 0.07 mg/kg to about 0.11 mg/kg, about 0.07 mg/kg to about 0.09 mg/kg, about 0.09 mg/kg to about 0.13 mg/kg, about 0.09 mg/kg to about 0.11 mg/kg, or about 0.11 mg/kg to about 0.13 mg/kg of infigratinib, or a pharmaceutically acceptable salt thereof. In certain embodiments, the method comprises administering to the pediatric patient about 0.015 mg/kg to about 0.13 mg/kg of infigratinib, or a pharmaceutically acceptable salt thereof.

In certain embodiments, the method comprises administering to the pediatric patient about 0.004 mg/kg, about 0.008 mg/kg, about 0.016 mg/kg, about 0.032 mg/kg, about 0.064 mg/kg, about 0.128 mg/kg, about 0.256 mg/kg, or about 0.51 mg/kg of infigratinib, or a pharmaceutically acceptable salt thereof.

In various embodiments, provided herein are methods of treating a pediatric patient suffering from a skeletal dysplasia such as hypochondroplasia or achondroplasia, comprising orally administering daily to the patient about 0.1 mg to about 8 mg of infigratinib, or a pharmaceutically acceptable salt thereof.

In certain embodiments, the method comprises administering about 0.1 mg to about 8 mg, about 0.2 mg to about 8 mg, about 0.3 mg to about 8 mg, about 0.4 mg to about 8 mg, about 0.5 mg to about 8 mg, about 1 mg to about 8 mg, about 1.5 mg to about 8 mg, about 2 mg to about 8 mg, about 2.5 mg to about 8 mg, about 3 mg to about 8 mg, about 3.5 mg to about 8 mg, about 4 mg to about 8 mg, about 4.5 mg to about 8 mg, about 5 mg to about 8 mg, about 5.5 mg to about 8 mg, about 6 mg to about 8 mg, about 6.5 mg to about 8 mg, about 7 mg to about 8 mg, about 7.5 mg to about 8 mg, about 0.1 mg to about 7.5 mg, about 0.1 mg to about 7 mg, about 0.1 mg to about 6.5 mg, about 0.1 mg to about 6 mg, about 0.1 mg to about 5.5 mg, about 0.1 mg to about 5 mg, about 0.1 mg to about 4.5 mg, about 0.1 mg to about 4 mg, about 0.1 mg to about 3.5 mg, about 0.1 mg to about 3 mg, about 0.1 mg to about 2.5 mg, about 0.1 mg to about 2 mg, about 0.1 mg to about 1.5 mg, about 0.1 mg to about 1 mg, about 0.1 mg to about 0.5 mg, about 0.1 mg to about 0.4 mg, about 0.1 mg to about 0.3 mg, about 0.1 mg to about 0.2 mg, about 0.2 mg to about 7.5 mg, about 0.2 mg to about 7 mg, about 0.2 mg to about 6.5 mg, about 0.2 mg to about 6 mg, about 0.2 mg to about 5.5 mg, about 0.2 mg to about 5 mg, about 0.2 mg to about 4.5 mg, about 0.2 mg to about 4 mg, about 0.2 mg to about 3.5 mg, about 0.2 mg to about 3 mg, about 0.2 mg to about 2.5 mg, about 0.2 mg to about 2 mg, about 0.2 mg to about 1.5 mg, about 0.2 mg to about 1 mg, about 0.2 mg to about 0.5 mg, about 0.2 mg to about 0.4 mg, about 0.2 mg to about 0.3 mg, about 0.3 mg to about 7.5 mg, about 0.3 mg to about 7 mg, about 0.3 mg to about 6.5 mg, about 0.3 mg to about 6 mg, about 0.3 mg to about 5.5 mg, about 0.3 mg to about 5 mg, about 0.3 mg to about 4.5 mg, about 0.3 mg to about 4 mg, about 0.3 mg to about 3.5 mg, about 0.3 mg to about 3 mg, about 0.3 mg to about 2.5 mg, about 0.3 mg to about 2 mg, about 0.3 mg to about 1.5 mg, about 0.3 mg to about 1 mg, about 0.3 mg to about 0.5 mg, about 0.3 mg to about 0.4 mg, about 0.4 mg to about 7.5 mg, about 0.4 mg to about 7 mg, about 0.4 mg to about 6.5 mg, about 0.4 mg to about 6 mg, about 0.4 mg to about 5.5 mg, about 0.4 mg to about 5 mg, about 0.4 mg to about 4.5 mg, about 0.4 mg to about 4 mg, about 0.4 mg to about 3.5 mg, about 0.4 mg to about 3 mg, about 0.4 mg to about 2.5 mg, about 0.4 mg to about 2 mg, about 0.4 mg to about 1.5 mg, about 0.4 mg to about 1 mg, about 0.4 mg to about 0.5 mg, about 0.5 mg to about 7.5 mg, about 0.5 mg to about 7 mg, about 0.5 mg to about 6.5 mg, about 0.5 mg to about 6 mg, about 0.5 mg to about 5.5 mg, about 0.5 mg to about 5 mg, about 0.5 mg to about 4.5 mg, about 0.5 mg to about 4 mg, about 0.5 mg to about 3.5 mg, about 0.5 mg to about 3 mg, about 0.5 mg to about 2.5 mg, about 0.5 mg to about 2 mg, about 0.5 mg to about 1.5 mg, about 0.5 mg to about 1 mg, about 1.5 mg to about 7.5 mg, about 1.5 mg to about 7 mg, about 1.5 mg to about 6.5 mg, about 1.5 mg to about 6 mg, about 1.5 mg to about 5.5 mg, about 1.5 mg to about 5 mg, about 1.5 mg to about 4.5 mg, about 1.5 mg to about 4 mg, about 1.5 mg to about 3.5 mg, about 1.5 mg to about 3 mg, about 1.5 mg to about 2.5 mg, about 1.5 mg to about 2 mg, about 2 mg to about 7.5 mg, about 2 mg to about 7 mg, about 2 mg to about 6.5 mg, about 2 mg to about 6 mg, about 2 mg to about 5.5 mg, about 2 mg to about 5 mg, about 2 mg to about 4.5 mg, about 2 mg to about 4 mg, about 2 mg to about 3.5 mg, about 2 mg to about 3 mg, about 2 mg to about 2.5 mg, about 2.5 mg to about 7.5 mg, about 2.5 mg to about 7 mg, about 2.5 mg to about 6.5 mg, about 2.5 mg to about 6 mg, about 2.5 mg to about 5.5 mg, about 2.5 mg to about 5 mg, about 2.5 mg to about 4.5 mg, about 2.5 mg to about 4 mg, about 2.5 mg to about 3.5 mg, about 2.5 mg to about 3 mg, about 3 mg to about 7.5 mg, about 3 mg to about 7 mg, about 3 mg to about 6.5 mg, about 3 mg to about 6 mg, about 3 mg to about 5.5 mg, about 3 mg to about 5 mg, about 3 mg to about 4.5 mg, about 3 mg to about 4 mg, about 3 mg to about 3.5 mg, about 3.5 mg to about 7.5 mg, about 3.5 mg to about 7 mg, about 3.5 mg to about 6.5 mg, about 3.5 mg to about 6 mg, about 3.5 mg to about 5.5 mg, about 3.5 mg to about 5 mg, about 3.5 mg to about 4.5 mg, about 3.5 mg to about 4 mg, about 4 mg to about 7.5 mg, about 4 mg to about 7 mg, about 4 mg to about 6.5 mg, about 4 mg to about 6 mg, about 4 mg to about 5.5 mg, about 4 mg to about 5 mg, about 4 mg to about 4.5 mg, about 4.5 mg to about 7.5 mg, about 4.5 mg to about 7 mg, about 4.5 mg to about 6.5 mg, about 4.5 mg to about 6 mg, about 4.5 mg to about 5.5 mg, about 4.5 mg to about 5 mg, about 5 mg to about 7.5 mg, about 5 mg to about 7 mg, about 5 mg to about 6.5 mg, about 5 mg to about 6 mg, about 5 mg to about 5.5 mg, about 5.5 mg to about 7.5 mg, about 5.5 mg to about 7 mg, about 5.5 mg to about 6.5 mg, about 5.5 mg to about 6 mg, about 6 mg to about 7.5 mg, about 6 mg to about 7 mg, about 6 mg to about 6.5 mg, about 6.5 mg to about 7.5 mg, about 7 mg to about 7.5 mg, or about 7 mg to about 7.5 mg of infigratinib, or a pharmaceutically acceptable salt thereof. In certain embodiments, the method comprises administering about 0.1 mg to about 7 mg of infigratinib, or a pharmaceutically acceptable salt thereof.

In certain embodiments, the method comprises administering about 0.1 mg, about 0.2 mg, about 0.3 mg, about 0.4 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, or about 8 mg of infigratinib, or a pharmaceutically acceptable salt thereof.

In certain embodiments, the pediatric patient has a FGFR3 mutation. In certain embodiments, the FGFR3 mutation is a G380R mutation.

In certain embodiments, the infigratinib, or a pharmaceutically acceptable salt thereof, is administered in the form of a minitablet. In certain embodiments, the infigratinib, or a pharmaceutically acceptable salt thereof, is administered in the form of a minitablet as described herein. In certain embodiments, the infigratinib, or a pharmaceutically acceptable salt thereof, is administered as minitablets each having about 0.1 mg or about 1 mg of infigratinib, or a pharmaceutically acceptable salt thereof. In certain embodiments, the infigratinib, or a pharmaceutically acceptable salt thereof, is administered as minitablets each having 0.1 mg or 1 mg of infigratinib, or a pharmaceutically acceptable salt thereof.

In certain embodiments, the infigratinib is present in the minitablets as infigratinib monophosphate. In certain embodiments, the infigratinib monophosphate is present as an anhydrous crystalline form. In certain embodiments, the anhydrous crystalline form of infigratinib monophosphate is characterized by an XRPD peak (2 theta) at 15.0°±0.2°.

In certain embodiments, after daily administration of the dose of infigratinib, or a pharmaceutically acceptable salt thereof, the pediatric patient exhibits increased height velocity relative to baseline. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% increase in height velocity relative to baseline.

In certain embodiments, after daily administration of the dose of infigratinib, or a pharmaceutically acceptable salt thereof, the pediatric patient exhibits an increase, relative to baseline, in an anthropometric measurement selected from the group consisting of standing height, sitting height, upper and lower arm length, thigh length, knee height, arm span, and combinations thereof. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% increase, relative to baseline, in an anthropometric measurement selected from the group consisting of standing height, sitting height, upper and lower arm length, thigh length, knee height, arm span, and combinations thereof.

In certain embodiments, after daily administration of the dose of infigratinib, or a pharmaceutically acceptable salt thereof, the pediatric patient exhibits an absolute increase in an anthropometric measurement selected from the group consisting of standing height, sitting height, upper and lower arm length, thigh length, knee height, arm span, and combinations thereof. In certain embodiments, after daily administration of the dose of infigratinib, or a pharmaceutically acceptable salt thereof, the pediatric patient exhibits an absolute increase of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% in an anthropometric measurement selected from the group consisting of standing height, sitting height, upper and lower arm length, thigh length, knee height, arm span, and combinations thereof.

In certain embodiments, after daily administration of the dose of infigratinib, or a pharmaceutically acceptable salt thereof, the pediatric patient exhibits a decrease, relative to baseline, in weight. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, or 20% decrease, relative to baseline, in weight.

In certain embodiments, after daily administration of the dose of infigratinib, or a pharmaceutically acceptable salt thereof, the pediatric patient exhibits an absolute decrease in weight. In certain embodiments, after daily administration of the dose of infigratinib, or a pharmaceutically acceptable salt thereof, the pediatric patient exhibits an absolute decrease of 1%, 2%, 3%, 4%, 5%, 6%, 7%, 8%, 9%, 10%, 11%, 12%, 13%, 14%, 15%, 16%, 17%, 18%, 19%, or 20% in weight.

In certain embodiments, after daily administration of the dose of infigratinib, or a pharmaceutically acceptable salt thereof, the pediatric patient exhibits a proportional increase, relative to baseline, in head circumference. In certain embodiments, after daily administration of the dose of infigratinib, or a pharmaceutically acceptable salt thereof, the pediatric patient exhibits an absolute proportional increase in head circumference. For example, after daily administration of the dose of infigratinib, or a pharmaceutically acceptable salt thereof, the body may come into proportion to the head. In contrast, in the absence of treatment with infigratinib, or a pharmaceutically acceptable salt thereof, the patient may have macroencephaly.

In certain embodiments, after daily administration of the dose of infigratinib, or a pharmaceutically acceptable salt thereof, the pediatric patient exhibits a normalization, relative to baseline, of a body proportion measurement ratio. In certain embodiments, after daily administration of the dose of infigratinib, or a pharmaceutically acceptable salt thereof, the pediatric patient exhibits an absolute normalization of a body proportion measurement ratio. For example, after daily administration of the dose of infigratinib, or a pharmaceutically acceptable salt thereof, the limbs could grow to be more proportionate compared to the trunk. In contrast, in the absence of treatment with infigratinib, or a pharmaceutically acceptable salt thereof, the patient may have limbs that are disproportionately short compared to the trunk.

In certain embodiments, the body proportion measurement ratio is selected from the group consisting of upper to lower body segment ratio, upper arm to forearm ratio, upper leg to lower leg length ratio, arm span to standing height ratio, head circumference to standing height ratio, and combinations thereof.

In certain embodiments, after daily administration of the dose of infigratinib, or a pharmaceutically acceptable salt thereof, the pediatric patient exhibits an increase, relative to baseline, in a biomarker of bone turnover selected from the group consisting of type X collagen degradation fragment, collagen X marker, and combinations thereof. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% increase, relative to baseline, in a biomarker of bone turnover selected from the group consisting of type X collagen degradation fragment, collagen X marker, and a combination thereof.

In certain embodiments, after daily administration of the dose of infigratinib, or a pharmaceutically acceptable salt thereof, the pediatric patient exhibits an increase, relative to baseline, in mobility. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% increase, relative to baseline, in mobility.

In certain embodiments, after daily administration of the dose of infigratinib, or a pharmaceutically acceptable salt thereof, the pediatric patient exhibits a decrease, relative to baseline, in the number of episodes of otitis media. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% decrease, relative to baseline, in the number of episodes of otitis media.

In certain embodiments, after daily administration of the dose of infigratinib, or a pharmaceutically acceptable salt thereof, the pediatric patient exhibits a decrease, relative to baseline, in the number of episodes and/or severity of sleep apnea. In certain embodiments, after daily administration of the dose of infigratinib, or a pharmaceutically acceptable salt thereof, the pediatric patient exhibits a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100% decrease, relative to baseline, in the number of episodes and/or severity of sleep apnea.

In certain embodiments, after daily administration of the dose of infigratinib, or a pharmaceutically acceptable salt thereof, the pediatric patient exhibits an increase in quality of life, wherein quality of life is assessed using the Pediatric Quality of Life Inventory.

In certain embodiments, the pediatric patient is no more than 2 years of age, no more than 3 years of age, no more than 4 years of age, no more than 5 years of age, no more than 6 years of age, no more than 7 years of age, no more than 8 years of age, no more than 9 years of age, no more than 10 years of age, no more than 11 years of age, no more than 12 years of age, no more than 13 years of age, no more than 14 years of age, no more than 15 years of age, no more than 16 years of age, no more than 17 years of age, no more than 18 years of age, no more than 19 years of age, no more than 20 years of age, or no more than 21 years of age. In certain embodiments, the pediatric patient is no more than 12 years of age.

In certain embodiments, the pediatric patient is 2 to 21 years of age, 3 to 21 years of age, 4 to 21 years of age, 5 to 21 years of age, 6 to 21 years of age, 7 to 21 years of age, 8 to 21 years of age, 9 to 21 years of age, 10 to 21 years of age, 11 to 21 years of age, 12 to 21 years of age, 13 to 21 years of age, 14 to 21 years of age, 15 to 21 years of age, 16 to 21 years of age, 17 to 21 years of age, 18 to 21 years of age, 19 to 21 years of age, 20 to 21 years of age, 2 to 20 years of age, 2 to 19 years of age, 2 to 18 years of age, 2 to 17 years of age, 2 to 16 years of age, 2 to 15 years of age, 2 to 14 years of age, 2 to 13 years of age, 2 to 12 years of age, 2 to 11 years of age, 2 to 10 years of age, 2 to 9 years of age, 2 to 8 years of age, 2 to 7 years of age, 2 to 6 years of age, 2 to 5 years of age, 2 to 4 years of age, 2 to 3 years of age, 3 to 20 years of age, 3 to 19 years of age, 3 to 18 years of age, 3 to 17 years of age, 3 to 16 years of age, 3 to 15 years of age, 3 to 14 years of age, 3 to 13 years of age, 3 to 12 years of age, 3 to 11 years of age, 3 to 10 years of age, 3 to 9 years of age, 3 to 8 years of age, 3 to 7 years of age, 3 to 6 years of age, 3 to 5 years of age, 3 to 4 years of age, 4 to 20 years of age, 4 to 19 years of age, 4 to 18 years of age, 4 to 17 years of age, 4 to 16 years of age, 4 to 15 years of age, 4 to 14 years of age, 4 to 13 years of age, 4 to 12 years of age, 4 to 11 years of age, 4 to 10 years of age, 4 to 9 years of age, 4 to 8 years of age, 4 to 7 years of age, 4 to 6 years of age, 4 to 5 years of age, 5 to 20 years of age, 5 to 19 years of age, 5 to 18 years of age, 5 to 17 years of age, 5 to 16 years of age, 5 to 15 years of age, 5 to 14 years of age, 5 to 13 years of age, 5 to 12 years of age, 5 to 11 years of age, 5 to 10 years of age, 5 to 9 years of age, 5 to 8 years of age, 5 to 7 years of age, 5 to 6 years of age, 6 to years of age, 6 to 19 years of age, 6 to 18 years of age, 6 to 17 years of age, 6 to 16 years of age, 6 to 15 years of age, 6 to 14 years of age, 6 to 13 years of age, 6 to 12 years of age, 6 to 11 years of age, 6 to 10 years of age, 6 to 9 years of age, 6 to 8 years of age, 6 to 7 years of age, 7 to 20 years of age, 7 to 19 years of age, 7 to 18 years of age, 7 to 17 years of age, 7 to 16 years of age, 7 to 15 years of age, 7 to 14 years of age, 7 to 13 years of age, 7 to 12 years of age, 7 to 11 years of age, 7 to 10 years of age, 7 to 9 years of age, 7 to 8 years of age, 8 to 20 years of age, 8 to 19 years of age, 8 to 18 years of age, 8 to 17 years of age, 8 to 16 years of age, 8 to 15 years of age, 8 to 14 years of age, 8 to 13 years of age, 8 to 12 years of age, 8 to 11 years of age, 8 to 10 years of age, 8 to 9 years of age, 9 to 20 years of age, 9 to 19 years of age, 9 to 18 years of age, 9 to 17 years of age, 9 to 16 years of age, 9 to 15 years of age, 9 to 14 years of age, 9 to 13 years of age, 9 to 12 years of age, 9 to 11 years of age, 9 to 10 years of age, 10 to 20 years of age, 10 to 19 years of age, 10 to 18 years of age, 10 to 17 years of age, 10 to 16 years of age, 10 to 15 years of age, 10 to 14 years of age, 10 to 13 years of age, 10 to 12 years of age, 10 to 11 years of age, 11 to 20 years of age, 11 to 19 years of age, 11 to 18 years of age, 11 to 17 years of age, 11 to 16 years of age, 11 to 15 years of age, 11 to 14 years of age, 11 to 13 years of age, 11 to 12 years of age, 12 to 20 years of age, 12 to 19 years of age, 12 to 18 years of age, 12 to 17 years of age, 12 to 16 years of age, 12 to 15 years of age, 12 to 14 years of age, 12 to 13 years of age, 13 to 20 years of age, 13 to 19 years of age, 13 to 18 years of age, 13 to 17 years of age, 13 to 16 years of age, 13 to 15 years of age, 13 to 14 years of age, 14 to 20 years of age, 14 to 19 years of age, 14 to 18 years of age, 14 to 17 years of age, 14 to 16 years of age, 14 to 15 years of age, 15 to 20 years of age, 15 to 19 years of age, 15 to 18 years of age, 15 to 17 years of age, 15 to 16 years of age, 16 to 20 years of age, 16 to 19 years of age, 16 to 18 years of age, 16 to 17 years of age, 17 to 20 years of age, 17 to 19 years of age, 17 to 18 years of age, 18 to 20 years of age, 18 to 19 years of age, or 19 to 20 years of age. In certain embodiments, In certain embodiments, the pediatric patient is 3 to 11 years of age.

In certain embodiments, the pediatric patient is less than 3 years of age, less than 4 years of age, less than 5 years of age, less than 6 years of age, less than 7 years of age, less than 8 years of age, less than 9 years of age, less than 10 years of age, less than 11 years of age, less than 12 years of age, less than 13 years of age, less than 14 years of age, less than 15 years of age, less than 16 years of age, less than 17 years of age, less than 18 years of age, less than 19 years of age, less than 20 years of age, or less than 21 years of age. In certain embodiments, the pediatric patient is less than 8 years of age.

In certain embodiments, the pediatric patient is 2 years of age or older, 3 years of age or older, 4 years of age or older, 5 years of age or older, 6 years of age or older, 7 years of age or older, 8 years of age or older, 9 years of age or older, 10 years of age or older, 11 years of age or older, 12 years of age or older, 13 years of age or older, 14 years of age or older, 15 years of age or older, 16 years of age or older, 17 years of age or older, 18 years of age or older, 19 years of age or older, or 20 years of age or older. In certain embodiments, the pediatric patient is 8 years of age or older.

In certain embodiments, the methods provided herein further comprise administering an effective amount of a second therapeutic agent to the patient.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1: Synthesis of 3-(2,6-dichloro-3,5-dimethoxy-phenyl)-1-{6-4-(4-ethyl-piperazin-1-yl)-phenylaminol-pyrimidin-4-yl}-1-methyl-urea (infigratinib)

### Step A: Synthesis of N-4-4(4-ethyl-piperazin-1-yl)-phenyl-N′-methyl-pyrimidine-4,6-diamine

A mixture of 4-(4-ethylpiperazin-1-yl)-aniline (1 g, 4.88 mmol), (6-chloro-pyrimidin-4-yl)-methyl-amine (1.81 g, 12.68 mmol. 1.3 eq.), and 4N HCl in dioxane (15 mL) is heated in a sealed tube to 150° C. for 5 hours. The reaction mixture is concentrated, diluted with dichloromethane (DCM) and a saturated aqueous solution of sodium bicarbonate. The aqueous layer is separated and extracted with DCM. The organic phase is washed with brine, dried (sodium sulfate), filtered and concentrated. Purification of the residue by silica gel column chromatography (DCM/MeOH, 93:7) followed by trituration in diethyl ether affords the title compound as a white solid: ESI-MS: 313.2 [MH]⁺; t_{R}=1.10 min (gradient J); TLC: R_{f}=0.21 (DCM/MeOH, 93:7).

### Step B: Synthesis of 4-(4-ethylpiperazin-1-yl)-aniline

A suspension of 1-ethyl-4-(4-nitro-phenyl)-piperazine (6.2 g, 26.35 mmol) and Raney Nickel (2 g) in MeOH (120 mL) is stirred for 7 hours at RT, under a hydrogen atmosphere. The reaction mixture is filtered through a pad of celite and concentrated to afford 5.3 g of the title compound as a violet solid: ESI-MS: 206.1 [MH]⁺; TLC: R_{f}=0.15 (DCM/MeOH+1% NH₃^{aq}, 9:1).

### Step C: Synthesis of 1-ethyl-4-(4-nitro-phenyl)-piperazine

A mixture of 1-bromo-4-nitrobenzene (6 g, 29.7 mmol) and 1-ethylpiperazine (7.6 mL, 59.4 mmol, 2 eq.) is heated to 80° C. for 15 hours. After cooling to RT, the reaction mixture is diluted with water and DCM/MeOH, 9:1. The aqueous layer is separated and extracted with DCM/MeOH, 9:1. The organic phase is washed with brine, dried (sodium sulfate), filtered and concentrated. Purification of the residue by silica gel column chromatography (DCM/MeOH+1% NH₃^{aq}, 9:1) affords 6.2 g of the title compound as a yellow solid: ESI-MS: 236.0 [MH]⁺; t_{R}=2.35 min (purity: 100%, gradient J); TLC: R_{f}=0.50 (DCM/MeOH+1% NH₃^{aq}, 9:1).

### Step D: Synthesis of (6-chloro-pyrimidin-4-yl)-methyl-amine

This material was prepared by a modified procedure published in the literature (J. Appl. Chem. 1955, 5, 358): To a suspension of commercially available 4,6-dichloropyrimidine (20 g, 131.6 mmol, 1.0 eq.) in isopropanol (60 mL) is added 33% methylamine in ethanol (40.1 mL, 328.9 mmol, 2.5 eq.) at such a rate that the internal temperature does not rise above 50° C. After completion of the addition the reaction mixture was stirred for 1 hour at room temperature. Then, water (50 mL) is added and the suspension formed is chilled in an ice bath to 5° C. The precipitated product is filtered off, washed with cold isopropanol/water 2:1 (45 mL) and water. The collected material is vacuum dried over night at 45° C. to afford the title compound as colorless powder: t_{R}=3.57 min (purity: >99%, gradient A), ESI-MS: 144.3/146.2 [MH]⁺.

### Step E: Synthesis of 3-(2,6-dichloro-3,5-dimethoxy-phenyl)-1-{6-4-(4-ethyl-piperazin-1-yl)-phenylaminol-pyrimidin-4-yl}-1-methyl-urea

The title compound was prepared by adding 2,6-dichloro-3,5-dimethoxyphenyl-isocyanate (1.25 eq.) to a solution of N-4-(4-ethyl-piperazin-1-yl)-phenyl)-N′-methyl-pyrimidine-4,6-diamine (2.39 g, 7.7 mmol, 1 eq.) in toluene and stirring the reaction mixture for 1.5 hours at reflux. Purification of the crude product by silica gel column chromatography (DCM/MeOH+1% NH₃^{aq}, 95:5) affords the title compound as a white solid: ESI-MS: 560.0/561.9 [MH]⁻; t_{R}=3.54 min (purity: 100%, gradient J); TLC: R_{f}=0.28 (DCM/MeOH+1% NH₃^{aq}, 95:5). Analysis: C₂₆H₃₁N₇O₃Cl₂, calc. C, 55.72%; H, 5.57%; N, 17.49%; O, 8.56%; Cl, 12.65%. Found C, 55.96%: H, 5.84%: N, 17.17%; O, 8.46%; Cl, 12.57%.

### Example 2: Synthesis of the Monophosphate Salt Form A of 3-(2,6-dichloro-3,5-dimethoxy-phenyl)-1-{6-4-(4-ethyl-piperazin-1-yl)-phenylaminol-pyrimidin-4-yl}-1-methyl-urea (BGJ398)

To a round bottom flask was added 3-(2,6-dichloro-3,5-dimethoxyphenyl)-1-(6-4-(4-ethylpiperazin-1-yl)phenylaminol-pyrimidine-4-yl)-1-methyl-urea (134 g, 240 mmol) and isopropanol (IPA) (2000 mL). The suspension was stirred and heated to 50° C. and a solution of phosphoric acid (73.5 g, 750 mmol) in water (2000 mL) added to it portions. The mixture was stirred at 60° C. for 30 minutes and filtered through a polypropylene pad. The pad was washed with warm IPA/water (1:1, 200 mL) and the filtrates were combined. To this clear solution, IPA (6000 mL) was added and the mixture was stirred under reflux for 20 minutes, cooled slowly to room temperature (25° C.), and stirred for 24 hours. The white salt product was collected by filtration, washed with IPA (2×500 mL) and dried in the oven at 60° C. under reduced pressure for two days to provide the anhydrous crystalline monophosphate salt (110 g). Yield 70%. Purity>98% by HPLC. Analysis: C₂₆H₃₄N₇O₇Cl₂P, calc. C, 47.42%; H, 5.20%; N, 14.89%; O, 17.01%; Cl, 10.77%; P. 4.70%. Found C, 47.40%; H, 5.11%: N, 14.71%; O, 17.18%: Cl, 10.73%; P, 4.87%.

### Example 3: 0.1 mg and 1 mg Dose Infigratinib Monophosphate Minitablets

In the following example, the manufacturing process is outlined for all exemplified dosage strengths.

The corresponding amounts of the ingredients are provided in the formulas under Examples 3.1, 3.2, 3.3, and 3.4 below.

### Manufacture of the Minitablets

For the 0.1 mg and 1.0 mg infigratinib minitablets with their formulations listed in Example 3.1 (Table 1) and Example 3.3 (Table 3), respectively, the manufacturing process consists of a wet granulation, blending, and compression at an example batch size of 5000 g with the following steps:
Add povidone to the dispensed purified water in a container and mix until povidone dissolves to form a binder solution.
Pass the intra-granular mannitol, microcrystalline cellulose and crospovidone through a #30 mesh screen Mannitol and microcrystalline cellulose are to be used in 4 portions and 3 portions, respectively.
Charge the mannitol (portion 1) and microcrystalline cellulose (portion 1) into a high shear granulator and mix for 5 minutes with the impeller speed at 100 rpm.
Pass the milled infigratinib phosphate (BGJ398 API) and mannitol (portion 2) through a #60 mesh screen for 0.1 mg infigratinib minitablets or a #50 mesh screen for 1.0 mg infigratinib minitablets.
Add the screened API, mannitol (portion 2) above and MCC (portion 2) into the high shear granulator and mix for 5 minutes with the impeller speed at 100 rpm.
Add mannitol (portion 3) into the high shear granulator and mix for 5 minutes with the impeller speed at 100 rpm.
Add the remaining mannitol, microcrystalline cellulose and intra-granular crospovidone into the high shear granulator and mix for 5 minutes with the impeller speed at 150 rpm.
Turn the high shear granulator “ON” with impeller speed at 150 rpm and spray the binder solution in approximately one minute.
After completion of binder solution addition, mix the wet mass for 30 seconds with the impeller speed at 150 rpm.
Mix the wet mass for additional 30 seconds with the impeller speed at 150 rpm and chopper speed at 1500 rpm.
Dry the wet granules in an appropriate size of fluid bed dryer at inlet air temperature at approximately 60° C. and air volume of approximately 250 m³/h until LOD is NMT 2%.
Mill the dried granules through Quadro Comil equipped with 2A032R screen at speed of ˜1500 rpm.
Pass the extra-granular crospovidone through a #30 mesh screen and FD&C Blue #1/Brilliant Blue FCF Aluminum Lake through a #80 mesh screen (FD&C Blue #1 for 1.0 mg infigratinib minitablets only).
Load the milled granules, extra-granular crospovidone and FD&C Blue #1/Brilliant Blue FCF Aluminum Lake (FD&C Blue #1 for 1.0 mg infigratinib minitablets only) into an appropriate size of bin blender and mix for 5 minutes at 26 rpm.
Add the screened magnesium stearate through a #40 mesh screen into the blender, mix for 5 minutes at 26 rpm and discharge as final blend.
Compress the final blend using appropriate press into minitablets using 2.2 mm round tooling.

For the 0.1 mg infigratinib minitablets with the formulation in Example 3.1 (Table 2), the manufacturing process consists of dry blending and compression at an example batch size of 5000 g with the following steps:
Screen the milled API through a #50 mesh screen, dispense the screened API into double polyethene bag lined container. Screen mannitol, microcrystalline cellulose, croscarmellose sodium through a #30 mesh screen. Divide the screened mannitol into 3 portions and microcrystalline cellulose into 2 portions.
Add the mannitol (portion 1) into the bag containing the screened milled API and mix for 1 minute manually.
Transfer the mixture above to an appropriate size of V-blender and blend for 10 minutes.
Add the screened microcrystalline cellulose (portion 1) into the blender and blend for 10 minutes.
Discharge and screen the blend above through a #30 mesh screen and load the blend back into the blender.
Add the screened mannitol (portion 2) into the blender and blend for 10 minutes.
Discharge the blend and screen through a #30 mesh screen and load the blend back into the blender.
Add the screened microcrystalline cellulose (portion 2) into the blender and blend for 10 minutes.
Discharge the blend from blender and screen the blend through a #30 mesh, then load the blend back to the blender.
Add the screened sodium croscarmellose into the blender and blend for 10 minutes
Pass the magnesium stearate through a #40 mesh screen and load it to the blender. Blend for 3 minutes at 21 rpm, and discharge as final blend.
Compress the final blend into minitablets using 2 mm round tooling.

For the 1.0 mg infigratinib minitablets with the formulation listed in Example 3.4 (Table 4), the manufacturing process consists of dry granulation, blending and compression at an example batch size of 5000 g with the following steps:
Screen the milled API through a #50 mesh screen, dispense the screened API into double polyethene bag lined container. Screen the intra-granular part of mannitol, microcrystalline cellulose, croscarmellose sodium through a #30 mesh screen and screen FD&C Blue #1/Brilliant Blue FCF Aluminum through a #50 mesh screen into a suitable labeled polybag lined container separately.
Load the intra-granular portion of mannitol, milled API, FD&C Blue #1/Brilliant Blue FCF Aluminum, microcrystalline cellulose and croscarmellose sodium to an appropriate size of V-blender, and blend for 10 minutes at 21 rpm.
Discharge the blend and screen the blend using a #30 mesh screen into a LDPE bag lined container. Load the screened blend into the blender and blend for 5 minutes at 21 rpm.
Pass the magnesium stearate through a #40 mesh screen and collect in the stainless-steel pan. Add the screened magnesium stearate to the blender. Blend for 3 minutes at 21 rpm.
Granulate the blend through the roller compactor to achieve a continuous stream of brittle ribbons. Collect the milled granules into tared double PE bag lined containers.
Screen the extra-granular part of microcrystalline cellulose and croscarmellose sodium using a #30 mesh screen.
Charge half of the dry granules, extra-granular microcrystalline cellulose, extra-granular croscarmellose sodium and rest of dry granules into an appropriate V-blender, and blend for 7 minutes at 21 rpm:
Screen the extra-granular magnesium stearate through a #40 mesh screen, add to the V-blender, blend for 5 minutes at 21 rpm and discharge as final blend.
Compress the final blend into minitablets using 2 mm round tooling.

### Example 3.1

TABLE 1
Formula for 0.1 mg Dosage Strength

0.1 mg Infigratinib

| | | |
|---|---|---|
| Ingredient | % w/w | mg/tab |

Intra-Granular

| | | |
|---|---|---|
| Milled BGJ398 API* | 1.68 | 0.1175 |
| Mannitol | 49.82 | 3.4875 |
| Microcrystalline Cellulose | 35.00 | 2.45 |
| Povidone | 7.00 | 0.49 |
| Crospovidone | 4.00 | 0.28 |
| Purified Water** | — | — |

Extra-Granular

| | | |
|---|---|---|
| Crospovidone | 2.00 | 0.14 |
| Magnesium Stearate | 0.50 | 0.035 |
| Total | 100 | 7.0 |

*the salt factor is 1.175;
**Purified water USP 28% w/w removed during drying process.

### Example 3.2

TABLE 2
Formula for 0.1 mg Dosage Strength

| Component | % w/w | mg/tab |
|---|---|---|
| Milled BGJ398 API* | 1.68 | 0.1175 |
| Mannitol | 57.57 | 4.0299 |
| Microcrystalline Cellulose | 35.00 | 2.4500 |
| Croscarmellose Sodium | 5.00 | 0.3500 |
| Magnesium Stearate | 0.75 | 0.0525 |
| Total | 100.00 | 7.00 |

*the salt factor is 1.175.

TABLE 3
Formula for 1 mg Dosage Strength

1.0 mg Infigratinib

| | | |
|---|---|---|
| Ingredient | % w/w | mg/tab |

Intra-Granular

| | | |
|---|---|---|
| Milled BGJ398 API* | 10.68 | 1.175 |
| Mannitol | 35.52 | 3.907 |
| Microcrystalline Cellulose | 40.00 | 4.40 |
| Povidone | 7.00 | 0.77 |
| Crospovidone | 4.00 | 0.44 |
| Purified Water** | — | — |

Extra-Granular

| | | |
|---|---|---|
| Crospovidone | 2.00 | 0.22 |
| FD&C Blue #1/Brilliant Blue | 0.05 | 0.006 |
| FCF Aluminum Lake | | |
| Magnesium Stearate | 0.75 | 0.083 |
| Total | 100 | 11.0 |

*the salt factor is 1.175;
**Purified water USP 28% w/w removed during drying process.

### Example 3.3

### Example 3.4

TABLE 4
Formula for 1 mg Dosage Strength

| | | |
|---|---|---|
| Component | % w/w | mg/tab |

Intra-Granular

| | | |
|---|---|---|
| Milled BGJ398 API* | 16.79 | 1.1750 |
| Mannitol | 42.16 | 2.9515 |
| Microcrystalline Cellulose | 25.00 | 1.7500 |
| Croscarmellose Sodium | 3.50 | 0.2450 |
| FD&C Blue #1/Brilliant Blue | 0.05 | 0.0035 |
| FCF Aluminum Lake | | |
| Magnesium Stearate | 0.50 | 0.0350 |

Extra-Granular

| | | |
|---|---|---|
| Microcrystalline Cellulose NF | 10.00 | 0.700 |
| Croscarmellose Sodium | 1.50 | 0.1050 |
| Magnesium Stearate NF | 0.50 | 0.0350 |
| Total | 100.0 | 7.0 |

*the salt factor is 1.175.

### Example 4: A Study of Oral Infigratinib Monophosphate (BGJ398) in Pediatric Patients with Achondroplasia

### Objectives

Dose Escalation: Primary Objective: Identify a dose of oral infigratinib, based on safety and efficacy evaluations, for children with achondroplasia (ACH) to be used for further study.

Dose Expansion: Primary Objective: Provide preliminary evidence of efficacy of oral infigratinib for the treatment of ACH, as assessed by change from baseline in height velocity in children with ACH.

Dose Escalation and Expansion: Secondary Objectives: Evaluate the safety and tolerability of oral infigratinib in children with ACH; evaluate changes from baseline in anthropometric parameters after administration of oral infigratinib; and evaluate the pharmacokinetic and pharmacodynamic (PK/PD) profile of infigratinib in children with ACH after administration of oral infigratinib. Exploratory Objective: evaluate changes in ACH disease burden.

### Methodology

Evaluate the safety, tolerability, and efficacy of infigratinib, a fibroblast growth factor receptor (FGFR) 1-3-selective tyrosine kinase inhibitor, in children 3 to 11 years of age with ACH who have previously been administered infigratinib for at least 6 months. The study includes dose escalation with extended treatment, and dose expansion.

Dose Escalation with Extended Treatment (total of 18 months treatment and follow-up): Eligible subjects 3 to 11 years of age are enrolled in ascending dose cohorts of approximately 10 subjects. The proportion of subjects<8 years and ≥8 years of age are approximately balanced between cohorts.

Up to 4 cohorts are planned. Each cohort commences after the prior dose has been deemed safe by the Data Review Committee (DRC) based on prespecified criteria (see Cohort Dose Escalation and Cohort Dose De-escalation below). In the United States (U.S.), subjects start enrollment in Cohorts 3 and 4 (i.e., subjects from the U.S. are not enrolled in Cohorts 1 or 2).

Subjects in each cohort are treated and followed up for 6 months at their assigned dose. After the 6-month study visit, subjects continue treatment for an additional 12 months (extended treatment period). To avoid long-term treatment with a possibly non- or subefficacious dose, subjects in Cohorts 1 and 2 have their dose increased to the next dose level at their 6-month and 12-month study visits, if no safety concerns are identified and their annualized height velocity does not increase at least 25% over baseline (a maximum of 2 dose increases is allowed) (not applicable in the U.S.). Dose increase at 12 months occurs if the dose identified for further study has not been determined by the time the subject reaches 12 months of treatment. Subjects in all cohorts may have their dose adjusted to the dose identified for further study, at the time this dose is determined. The dose identified for further study is selected based on thorough review of efficacy and safety findings from the dose escalation after all subjects have had the potential to complete 6 months of infigratinib treatment.

Dose Expansion (Total of 12 Months Treatment):

To support and confirm the dose/dose regimen identified for further study, 20 subjects are enrolled into the dose expansion and receive infigratinib treatment at the identified dose for 12 months.

All Subjects:

After completing study activities, all subjects may have the opportunity to enroll in an open-label long-term extension study (administered under a separate protocol) to assess the safety and efficacy of long-term administration of infigratinib in children with ACH.

Starting Dose and Determination of Other Cohort Doses

Starting Dose:

The starting dose in this Phase 2 study is based on the no observed adverse effect level (NOAEL) from a rat juvenile toxicity study with a 10-fold safety margin. In the U.S., the study will start in Cohort 3, corresponding to a dose that also accounts for estimation of pharmacologically active doses based on studies conducted in a mouse model that resembles achondroplasia.

Other Dose Cohorts:

Ascending dose cohorts initiate after safety of the prior cohort is confirmed by a Data Review Committee (DRC). Each successive dose is double the previous dose. In the U.S., the Cohort 4 dose is double the dose administered in Cohort 3.

De-escalation of a dose cohort is done for safety reasons based on prespecified criteria (see Cohort Dose Escalation and Cohort Dose De-escalation below).

Number of Subjects

A total of approximately 60 subjects are enrolled: 40 subjects in dose escalation with extended treatment, and 20 subjects in dose expansion.

Diagnosis and Main Criteria for Inclusion

Inclusion Criteria:
1. Signed informed consent by subject or parent(s) or legally authorized representative (LAR) and signed informed assent by the subject (when applicable).
2. 3 to 11 years of age (inclusive) at screening.
3. Diagnosis of ACH, documented clinically and confirmed by genetic testing.
4. At least a 6-month period of growth assessment in the PROPEL study (Protocol QBGJ398-001) before study entry.
5. Ambulatory and able to stand without assistance.
6. If a girl≥10 years of age, negative pregnancy test.
7. If sexually active, willing to use a highly effective method of contraception while taking study drug and for 3 months after the last dose of study drug.
8. Subjects and parent(s) or LAR are willing and able to comply with study visits and study procedures.
9. Able to swallow oral medication.
10. Willing to stop consumption of grapefruit, grapefruit juice, grapefruit hybrids, pomegranates, star fruits, pomelos, Seville oranges, or products containing juice of these fruits; and have not consumed these within 7 days before the first dose of study drug.

Exclusion Criteria:
1. Hypochondroplasia or short stature condition other than ACH (e.g., trisomy 21, pseudoachondroplasia, psychosocial short stature).
2. In females, having had their menarche.
3. Height<−2 or >+2 standard deviations for age and sex based on reference tables on growth in children with ACH (Horton 1978).
4. Annualized height velocity≤1.5 cm/year over a period≥6 months prior to screening.
5. Significant concurrent disease or condition that, in the view of the Investigator and/or Sponsor, would confound assessment of efficacy or safety of infigratinib, including but not limited to the following:
   cardiac or vascular disease
   significant electrocardiogram abnormalities such as evidence of a previous myocardial infarction, left ventricular hypertrophy, flat T waves (particularly in the inferior leads) or more than minor non-specific ST-T wave changes, QRS>90 msec, QT interval corrected using Fridericia's formula (QTcF)>440 msec, PR interval>170 msec; or complete right or left bundle branch block
   hyperthyroidism
   abnormal thyroid levels or recently diagnosed hypothyroidism that has not been stable on therapy for at least 3 months
   uncontrolled (HbA1c>9%) or insulin-requiring diabetes mellitus
   adrenal insufficiency
   autoimmune inflammatory disease
   inflammatory bowel disease
   severe sleep apnea requiring surgery or new use of continuous positive airway pressure (CPAP) machine (based on the screening sleep study)
6. Significant abnormality in screening laboratory results, including but not limited to the following:
   a. Hemoglobin<10.0 g/dL.
   b. Total bilirubin>1.5×upper limit of normal (ULN).
   c. AST/SGOT or ALT/SGPT>2×ULN.
   d. Calculated or measured creatinine clearance of <60 mL/min.
7. Current evidence of corneal or retinal disorder/keratopathy including, but not limited to, bullous/band keratopathy, corneal abrasion, inflammation/ulceration, or keratoconjunctivitis, confirmed by ophthalmic examination.
8. History and/or current evidence of extensive ectopic tissue calcification.
9. History of malignancy.
10. Currently using medications known to prolong the QT/QTc interval (within 7 days or 5 half-lives [whichever is longer] prior to the Screening Visit) or receiving treatment with agents that are known strong inducers or inhibitors of CYP3A4 and medications which increase serum phosphorus and/or calcium concentration. Subjects are not permitted to receive vitamin D analogues; medications that alter the pH of the gastrointestinal tract, including antacids, H2 antagonists (e.g., ranitidine), and proton-pump inhibitors (e.g., omeprazole); or enzyme-inducing anti-epileptic drugs, including carbamazepine, phenytoin, phenobarbital, and primidone.
11. Current evidence of endocrine alterations of calcium/phosphorus homeostasis:
   a. Inorganic phosphorus outside of normal limits.
   b. Total serum calcium (corrected) outside of normal limits.
12. Allergy to any components of the study drug.
13. Treatment with growth hormone, insulin-like growth factor 1 (IGF-1), or anabolic steroids in the previous 6 months or long-term treatment (>3 months) at any time.
14. Treatment with a C-type natriuretic peptide (CNP) analog, fibroblast growth factor (FGF) ligand trap, or treatment targeting FGFR inhibition at any time.
15. Regular long-term treatment (≥3 weeks) with supraphysiologic doses of glucocorticoid therapy (i.e., >15 mg/m²/day of hydrocortisone or equivalence) or treatment with glucocorticoids at anti-inflammatory doses for over 3 weeks within 6 months of the screening visit (low-dose ongoing inhaled steroid for asthma is acceptable).
16. Treatment with any other investigational product or investigational medical device for the treatment of ACH or short stature.
17. Previous limb-lengthening surgery or guided growth surgery.
18. Fracture within 6 months of screening (due to potential effects on bone biomarkers and bone morphology).

Test Product, Dose and Mode of Administration

Four dose cohorts are used:
Cohort 1: 0.016 mg/kg
Cohort 2: 0.032 mg/kg
Cohort 3: 0.064 mg/kg
Cohort 4: 0.128 mg/kg

In non-U.S. countries, infigratinib is provided for oral dosing with a starting dose (Cohort 1) of 0.016 mg/kg QD, which corresponds to one-tenth of the nonclinical NOAEL. In the U.S., the study starts in Cohort 3 (0.064 mg/kg), corresponding to a dose that also accounts for estimation of pharmacologically active doses based on studies conducted in a mouse model that resembles achondroplasia.

Endpoints

Dose Escalation: Primary Endpoint

Treatment-emergent adverse events (TEAEs) that lead to dose decrease or discontinuation. Change from baseline in height velocity (annualized to cm/year). (Baseline is defined as the annualized height velocity obtained from a minimum of 6 months of observation in the PROPEL study.)

Dose Expansion: Primary Endpoint

Change from baseline in height velocity (annualized to cm/year).

Dose Escalation and Dose Expansion: Secondary Endpoints

Safety evaluations by incidence, type, severity, and causality of adverse events (AEs), serious adverse events (SAEs), laboratory test results (urinalysis, chemistry, hematology), clinically significant changes in vital signs, physical examination (including ophthalmic and dental evaluation), electrocardiograms, and imaging.

Absolute height velocity (annualized to cm/year), expressed numerically and as Z-score in relation to non-ACH tables.

Absolute (expressed as absolute value and Z-score in relation to ACH and non-ACH standardized pediatric growth curves) and change from baseline in anthropometric parameters, including body proportions. Anthropometric measurements may include, but may not be limited to, standing height, sitting height, weight, head circumference, upper and lower arm length, thigh length, knee height, and arm span. Body proportion measurement ratios may include, but may not be limited to, upper to lower body segment ratio, upper arm to forearm length ratio, upper leg to lower leg length ratio, arm span to standing height ratio, and head circumference to standing height ratio.

PK parameters (e.g., Cₘₐₓ and tₘₐₓ)

Changes in PD parameters (biomarkers of bone turnover that may include type X collagen degradation fragment, collagen X marker [CXM]).

Dose Escalation and Dose Expansion: Exploratory Endpoint

Changes in disease-specific complications, such as changes in mobility (assessed by elbow, hip, and knee range of motion), changes in the number of episodes of otitis media per year, changes in number of episodes and/or severity of sleep apnea, and changes in quality of life [QoL] as assessed by PedsQL (generic core scale short form, child and parent reports).
Baseline for range of motion and PedsQL will correspond to the values obtained at the baseline visit.
Baseline for the number of episodes of otitis media will be the number of episodes recorded during the PROPEL study [expressed as episodes/year].
Baseline for sleep apnea, will correspond to the polysomnogram performed at screening to rule out severe sleep apnea].

Data Review Committee (DRC); Cohort Dose Escalation; Cohort Dose De-Escalation; Dose Decrease/Discontinuation for an Individual Subject

Data Review Committee (DRC)

This study utilizes a DRC that monitors subject safety and key efficacy data and provide recommendations to the Sponsor regarding dose escalation, de-escalation, and/or expansion of dose cohorts.

Cohort dose escalation and de-escalation is decided by the DRC based on the Bayesian optimal interval (BOIN) design (Liu 2015) with a target toxicity rate of 25%.

Cohort Dose Escalation

Each cohort commences after safety (ECG, vital signs, physical exams, TEAE assessment and clinical labs) of the prior dose cohort has been reviewed and confirmed by the DRC. During dose escalation, the opening of a new ascending dose cohort is decided by the DRC based on review of safety data from approximately 10 subjects in each cohort after they complete at least 4 weeks of treatment and safety assessments. If after at least 4 weeks of treatment, ≤ 1/10 subjects met a dose decrease/discontinuation criterion (see below: Dose Decrease/Discontinuation for an Individual Subject), and no other safety concern is identified by the DRC, the next dose cohort opens.

Cohort Dose De-Escalation

The need for a cohort dose de-escalation is determined by the DRC based on the safety assessment and incidence of TEAEs that leads to dose decrease/discontinuation for an individual subject (see below). At any point and after 3 subjects receive treatment in a cohort, if ≥30% of subjects in the cohort meet the dose decrease/discontinuation criteria, then enrollment in that and/or any higher dose cohort (if applicable) is paused and the DRC is convened. The DRC will determine if the dose of the current or higher cohort should be de-escalated (i.e., subjects in current cohort continue treatment at the next lower dose for efficacy assessment) or if treatment can continue at the same dose and/or if a cohort expansion is needed to continue evaluating the safety of that dose level.

Dose Decrease/Discontinuation for an Individual Subject

Although the DRC monitors subject safety and considers the number of subjects meeting the dose decrease/discontinuation criteria to determine whether a cohort dose escalation can proceed or if a dose de-escalation is needed at the cohort level, dose modifications in an individual subject is managed by the Investigator.

The following is considered an AE that requires dose reduction/discontinuation in an individual subject:
1. Phosphorus level>4.5 mg/dL (or age-adjusted upper level of normal for reporting laboratory), confirmed by a repeat value.
2. Calcium level>10.7 mg/dL (or age-adjusted upper level of normal for reporting laboratory), confirmed by a repeat value.
3. Grade 2 or higher related (as assessed by the Investigator) treatment-emergent AE.
4. Grade 1 or higher corneal toxicity.

Subjects who experience at least one of the above-described AEs should have their dose modified as described below:
Suspend dose in individual subject until
   Phosphorus and/or calcium levels return to normal values; or
   Grade≥2 treatment-related AEs decrease in severity to below Grade 2
   Abnormal ocular findings resolve
Reinitiate dosing at the next lower dose level
If the AE that led to dose suspension does not resolve within 4 weeks with adequate supportive care, the subject may be discontinued.

Subject Discontinuation/Withdrawal from the Study or Study Drug

Early withdrawal from the study or study drug may occur for any of the following reasons:
Subject (or parent/LAR) request, or withdrawal of consent.
AEs that led to dose suspension did not resolve within 4 weeks with adequate supportive care.
The Investigator considers that is in the subject's best interest to discontinue from the study drug or study, including but not limited to worsening of disproportionate growth, development of or worsening of tibial bowing, occurrence of infigratinib-related fracture of bone and growth plate, and worsening of elbow joint range of motion.

Subject reaches final height or near final height as defined by Tanner stage of puberty≥4 and growth velocity≤1.5 cm/year (Marshall 1969; Marshall 1970).
Subject has a height velocity of ≤1.5 cm/year over at least a 6-month period.
Subject develops a clinically significant condition that can confound the assessment of efficacy or safety or will require the treatment with a prohibited medication such as vitamin D analogues; medications that alter the pH of the gastrointestinal tract, including antacids, H2 antagonists (e.g., ranitidine), and proton-pump inhibitors (e.g., omeprazole); or enzyme-inducing anti-epileptic drugs, including carbamazepine, phenytoin, phenobarbital, and primidone, etc. (see Section 8.3 [main protocol]).
Female subject becomes pregnant.
Protocol deviation (at the Sponsor's discretion).
Study termination by the Sponsor.
Lost to follow up.

Statistical Methods

Sample Size

Dose escalation and de-escalation rules are based on the BOIN design with a target toxicity rate of 25%. Selection of the interval boundaries in Table 10 (main protocol) is based on a maximum toxicity of 15% for a subtherapeutic dose and a minimum toxicity of 35% for an overly toxic dose. In addition, if there is a ≥95% chance that the rate of TEAEs that lead to dose decrease or discontinuation is ≥25% based on observed data, then the current dose cohort is eliminated from the trial; if the first dose level is eliminated, the trial may be terminated or a lower dose may be evaluated according to DRC recommendation.

The selection of the dose for dose expansion is based on the assessment of approximately 10 subjects per cohort. If a true AE incidence is 25%, 10 subjects per cohort allow observation of at least one AE with 94.4% confidence. With 10 subjects per cohort, there is also a 62.5% chance of obtaining a 95% confidence interval (CI) for height velocity with a half-width that is at most 1.5 cm/year, assuming the change from baseline of height velocity follows a normal distribution and the standard deviation is 2 cm/year.

In dose expansion, approximately 20 subjects are enrolled at the selected dose level. An annualized height velocity increase of ≤0.5 cm/year is considered not clinically relevant and is used as the null hypothesis. Assuming an increase in height velocity of 2 cm/year after initiation of infigratinib treatment, with a standard deviation of 2 cm/year, 20 subjects provide approximately 88.9% power to demonstrate that treatment with infigratinib can increase the height velocity>0.5 cm/year at a one-sided significance level of 0.025.

Dose Escalation

For dose escalation, all analyses are performed separately for each dosing cohort based on the originally received dose and in total. Ongoing analyses are performed to support DRC reviews. The selection of the dose to explore in dose expansion are based on thorough review of safety and efficacy data after all subjects have had the potential to complete 6 months of infigratinib treatment.

Dose Expansion

Subjects enrolled in dose expansion are analyzed for both safety and efficacy. These data are used to make inferences about change from baseline in height velocity. Ongoing analyses may be performed, and the final analysis for dose expansion occurs after all subjects have had the opportunity to complete 12 months of treatment in dose expansion.

Statistical Analyses

All safety analyses are performed using the safety analysis set, defined as subjects who have received at least one dose of study drug. Analyses on growth parameter endpoints are performed for subjects who have a baseline and at least one post-baseline growth parameter assessment. Baseline and demographic variables are summarized. Safety summaries present AEs recorded through the last dose date +30 days. All TEAEs are summarized and listed. TEAEs that lead to dose decrease or discontinuation are summarized. Laboratory measures, changes to disease-specific complications, and surgical procedures are summarized.

For subjects enrolled in dose escalation, the change from baseline on annualized height velocity, in addition to weight, height, head circumference, and body proportions at baseline and post-baseline; and changes in these parameters, are summarized based on the first 6-month assessments. For assessments done after 6 months, the summaries are provided in 6-month intervals by the originally received dose.

For subjects enrolled in dose expansion, the change from baseline on annualized height velocity are tested using one-sample t-test to assess whether the increase is >0.5 cm/year. Descriptive statistics, including 95% confidence intervals, are also provided for height velocity parameters, in addition to weight, height, head circumference, and body proportions at baseline and post-baseline; and changes in these parameters from baseline.

Descriptive statistics are also provided to explore the association between biomarkers and height velocity. Assessments of disease-specific complications are summarized by visit.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method of treating achondroplasia or hypochondroplasia in a pediatric patient in need thereof, comprising orally administering daily to the pediatric patient about 0.01 milligrams per kilogram (mg/kg) to about 0.51 mg/kg of infigratinib, or a pharmaceutically acceptable salt thereof.

2. The method of claim 1, comprising administering to the pediatric patient about 0.01 mg/kg to about 0.3 mg/kg of infigratinib, or a pharmaceutically acceptable salt thereof.

3. The method of claim 1, comprising administering to the pediatric patient about 0.01 mg/kg to about 0.15 mg/kg of infigratinib, or a pharmaceutically acceptable salt thereof.

4. The method of claim 1, comprising administering to the pediatric patient about 0.015 mg/kg to about 0.13 mg/kg of infigratinib, or a pharmaceutically acceptable salt thereof.

5. The method of claim 1, comprising administering to the pediatric patient about 0.016 mg/kg, about 0.032 mg/kg, about 0.064 mg/kg, about 0.128 mg/kg, about 0.256 mg/kg, or about 0.51 mg/kg of infigratinib, or a pharmaceutically acceptable salt thereof.

6. A method of treating a pediatric patient suffering from achondroplasia or hypochondroplasia, comprising orally administering daily to the patient about 0.1 mg to about 8 mg of infigratinib, or a pharmaceutically acceptable salt thereof.

7. The method of claim 6, comprising administering to the pediatric patient about 1 mg to about 7 mg of infigratinib, or a pharmaceutically acceptable salt thereof.

8. The method of any one of claims 1-7, wherein the pediatric patient has a FGFR3 mutation.

9. The method of claim 8, wherein the FGFR3 mutation is a G380R mutation.

10. The method of any one of claims 1-9, wherein the infigratinib, or a pharmaceutically acceptable salt thereof, is administered as minitablets each having 0.1 mg or about 1 mg of infigratinib, or a pharmaceutically acceptable salt thereof.

11. The method of claim 10, wherein the infigratinib is present in the minitablets as infigratinib monophosphate.

12. The method of claim 11, wherein the infigratinib monophosphate is present as an anhydrous crystalline form.

13. The minitablet of claim 12, wherein the anhydrous crystalline form of infigratinib monophosphate is characterized by an XRPD peak (2 theta) at 15.0°±0.2°.

14. The method of any one of claims 1-13, wherein, after daily administration of the dose of infigratinib, or a pharmaceutically acceptable salt thereof, the pediatric patient exhibits increased height velocity relative to baseline.

15. The method of any one of claims 1-13, wherein, after daily administration of the dose of infigratinib, or a pharmaceutically acceptable salt thereof, the pediatric patient exhibits an increase in absolute height velocity.

16. The method of any one of claims 1-13, wherein, after daily administration of the dose of infigratinib, or a pharmaceutically acceptable salt thereof, the pediatric patient exhibits an increase, relative to baseline, in an anthropometric measurement selected from the group consisting of standing height, sitting height, upper and lower arm length, thigh length, knee height, arm span, and combinations thereof.

17. The method of any one of claims 1-13, wherein, after daily administration of the dose of infigratinib, or a pharmaceutically acceptable salt thereof, the pediatric patient exhibits an absolute increase in an anthropometric measurement selected from the group consisting of standing height, sitting height, upper and lower arm length, thigh length, knee height, arm span, and combinations thereof.

18. The method of any one of claims 1-13, wherein, after daily administration of the dose of infigratinib, or a pharmaceutically acceptable salt thereof, the pediatric patient exhibits a decrease, relative to baseline, in weight.

19. The method of any one of claims 1-13, wherein, after daily administration of the dose of infigratinib, or a pharmaceutically acceptable salt thereof, the pediatric patient exhibits an absolute decrease in weight.

20. The method of any one of claims 1-13, wherein, after daily administration of the dose of infigratinib, or a pharmaceutically acceptable salt thereof, the pediatric patient exhibits a proportional increase, relative to baseline, in head circumference.

21. The method of any one of claims 1-13, wherein, after daily administration of the dose of infigratinib, or a pharmaceutically acceptable salt thereof, the pediatric patient exhibits an absolute proportional increase in head circumference.

22. The method of any one of claims 1-13, wherein, after daily administration of the dose of infigratinib, or a pharmaceutically acceptable salt thereof, the pediatric patient exhibits a normalization, relative to baseline, of a body proportion measurement ratio.

23. The method of any one of claims 1-13, wherein, after daily administration of the dose of infigratinib, or a pharmaceutically acceptable salt thereof, the pediatric patient exhibits an absolute normalization of a body proportion measurement ratio.

24. The method of claim 22 or 23, wherein the body proportion measurement ratio is selected from the group consisting of upper to lower body segment ratio, upper arm to forearm ratio, upper leg to lower leg length ratio, arm span to standing height ratio, head circumference to standing height ratio, and combinations thereof.

25. The method of any one of claims 1-13, wherein, after daily administration of the dose of infigratinib, or a pharmaceutically acceptable salt thereof, the pediatric patient exhibits an increase, relative to baseline, in a biomarker of bone turnover selected from the group consisting of type X collagen degradation fragment, collagen X marker, and combinations thereof.

26. The method of any one of claims 1-13, wherein, after daily administration of the dose of infigratinib, or a pharmaceutically acceptable salt thereof, the pediatric patient exhibits an increase, relative to baseline, in mobility.

27. The method of any one of claims 1-13, wherein, after daily administration of the dose of infigratinib, or a pharmaceutically acceptable salt thereof, the pediatric patient exhibits a decrease, relative to baseline, in the number of episodes of otitis media.

28. The method of any one of claims 1-13, wherein, after daily administration of the dose of infigratinib, or a pharmaceutically acceptable salt thereof, the pediatric patient exhibits a decrease, relative to baseline, in the number of episodes and/or severity of sleep apnea.

29. The method of any one of claims 1-13, wherein, after daily administration of the dose of infigratinib, or a pharmaceutically acceptable salt thereof, the pediatric patient exhibits an increase in quality of life, wherein quality of life is assessed using the Pediatric Quality of Life Inventory.

30. The method of any one of claims 1-29, wherein the pediatric patient is no more than 12 years of age.

31. The method of any one of claims 1-30, wherein the pediatric patient is 3 to 11 years of age.

32. The method of any one of claims 1-30, wherein the pediatric patient is less than 8 years of age.

33. The method of any one of claims 1-29, wherein the pediatric patient is 8 years of age or older.

34. A minitablet for orally delivering 1 mg of infigratinib, comprising:
infigratinib monophosphate, in an amount to deliver 1 mg of infigratinib, and a pharmaceutically acceptable excipient.

35. A minitablet for orally delivering 0.1 mg of infigratinib, comprising:
infigratinib monophosphate, in an amount to deliver 0.1 mg of infigratinib, and a pharmaceutically acceptable excipient.

36. The minitablet of claim 34 or 35, wherein the pharmaceutically acceptable excipient is selected from the group consisting of mannitol, microcrystalline cellulose, a polyvinylpyrrolidone, a cross-linked polyvinylpyrrolidone, magnesium stearate, croscarmellose sodium, and combinations thereof.

37. A minitablet for oral delivery, comprising:
about 1% w/w to about 20% w/w of infigratinib monophosphate; and
about 30% w/w to about 95% w/w of a filler.

38. The minitablet of claim 37, wherein the filler is selected from the group consisting of microcrystalline cellulose, lactose, mannitol, and combinations thereof.

39. The minitablet of claim 37 or 38, wherein the filler comprises microcrystalline cellulose and mannitol.

40. The minitablet of any one of claims 37-39, further comprising about 5% w/w to about 10% w/w of a binder.

41. The minitablet of claim of 40, wherein the binder is selected from the group consisting of a sugar, a gelatin, a natural gum, sorbitol, maltodextrin, an alginate, an alginate derivative, a polyvinylpyrrolidone, a cellulose, a cellulose derivative, and combinations thereof.

42. The minitablet of any one of claims 37-41, further comprising about 5% w/w to about 10% w/w of a disintegrant.

43. The minitablet of claim of 40, wherein the disintegrant is selected from the group consisting of a starch, a starch derivative, a clay, a cross-linked cellulose, a cross-linked cellulose derivative, a cross-linked polyvinylpyrrolidone, and combinations thereof.

44. A minitablet for oral delivery, comprising:
about 1% w/w to about 20% w/w of infigratinib monophosphate;
about 30% w/w to about 60% w/w of mannitol; and
about 30% w/w to about 45% w/w of microcrystalline cellulose.

45. The minitablet of claim 44, comprising about 10% w/w to about 20% w/w of infigratinib monophosphate.

46. The minitablet of claim 44, comprising about 1% w/w to about 5% w/w of infigratinib monophosphate.

47. The minitablet of claim 44, comprising about 30% w/w to about 45% w/w of mannitol.

48. The minitablet of claim 44, comprising about 45% w/w to about 60% w/w of mannitol.

49. The minitablet of any one of claims 44-48, comprising about 35% w/w to about 40% w/w of microcrystalline cellulose.

50. A minitablet for oral delivery, comprising:
about 10% w/w to about 20% w/w of infigratinib monophosphate;
about 30% w/w to about 45% w/w of mannitol; and
about 30% w/w to about 40% w/w of microcrystalline cellulose.

51. A minitablet for oral delivery, comprising:
about 1% w/w to about 5% w/w of infigratinib monophosphate;
about 45% w/w to about 60% w/w of mannitol; and
about 30% w/w to about 40% w/w of microcrystalline cellulose.

52. The minitablet of claim 50 or 51, further comprising about 5% w/w to about 10% w/w of a polyvinylpyrrolidone.

53. The minitablet of any one of claims 50-52, further comprising about 5% w/w to about 10% w/w of a cross-linked polyvinylpyrrolidone.

54. The minitablet of any one of claims 50-53, further comprising about 2% w/w to about 6% w/w of croscarmellose sodium.

55. A minitablet for oral delivery, comprising:
about 1.2 mg of infigratinib monophosphate;
about 3 mg to about 4 mg of mannitol; and
about 2 mg to about 4.4 mg of microcrystalline cellulose.

56. The minitablet of claim 55, further comprising about 0.6 mg to about 0.8 mg of a polyvinylpyrrolidone.

57. The minitablet of claim 55, further comprising about 0.5 mg to about 0.7 mg of a cross-linked polyvinylpyrrolidone.

58. A minitablet for oral delivery, comprising:
about 0.12 mg of infigratinib monophosphate;
about 3 mg to about 4 mg of mannitol; and
about 2 mg to about 3 mg of microcrystalline cellulose.

59. The minitablet of claim 58, further comprising about 0.4 mg to about 0.6 mg of a polyvinylpyrrolidone.

60. The minitablet of claim 58 or 59, further comprising about 0.3 mg to about 0.5 mg of a cross-linked polyvinylpyrrolidone.

61. The minitablet of any one of claims 55-60, further comprising about 0.2 mg to about 0.4 mg of croscarmellose sodium.

62. The minitablet of any one of claims 34-61, wherein the infigratinib monophosphate is present as an anhydrous crystalline form.

63. The minitablet of claim 62, wherein the anhydrous crystalline form of infigratinib monophosphate is characterized by an XRPD peak (2 theta) at 15.0°±0.2°.
